(19) United States
(12) Patent Application Publication
(10) Pub. No.: US 2023/0390057 A1
(43) Pub. Date: Dec. 7, 2023

(54) SPLIT-FRAME CARDIAC VALVES

(71) Applicant: Open Stent Solution SAS, Dury (FR)

(72) Inventors: Andrea Guidotti, Zurich (CH); David Zarbatany, Laguna Niguel, CA (US); Ricardo Roman, Chula Vista, CA (US); Georgios Stefopoulos, Zurich (CH); Pietro Gozzoli, Zurich (CH)

(73) Assignee: Open Stent Solution SAS, Dury (FR)

(21) Appl. No.: 18/332,403

(22) Filed: Jun. 9, 2023

Related U.S. Application Data
(63) Continuation of application No. PCT/IB2021/061480, filed on Dec. 9, 2021.
(60) Provisional application No. 63/123,528, filed on Dec. 10, 2020.

(51) Int. Cl. A61F 2/24

(52) U.S. Cl. CPC A61F 2/2418; A61F 2/2427; A61F 2250/001; A61F 2250/0008; A61F 2230/0069

(57) ABSTRACT

A split-frame cardiac valve (20) is configured to assume a radially compressed configuration for transcatheter delivery and a radially expanded configuration for anchoring to a native cardiac valve annulus. The split-frame cardiac valve (20) includes circumferential frame segments (22) including respective stents (24) and prosthetic leaflets (30). When the split-frame cardiac valve (20) is in the radially expanded configuration: (a) each of the stents (24) surrounds less than 360 degrees of a central longitudinal axis (26) of the split-frame cardiac valve (20), (b) the circumferential frame segments (22) are slidingly coupled to one another so as to be axially slidable with respect to one another in and out of axial alignment with one another, and (c) when the circumferential frame segments (22) are axially aligned with one another, the stents (24) collectively define a tubular stent (32) that entirely surrounds the central longitudinal axis (26).

### CROSS-REFERENCE TO RELATED APPLICATIONS

The present application is a continuation of PCT Application PCT/IB2021/061480, filed Dec. 9, 2021, which claims priority from U.S. Provisional Application 63/123,528, filed Dec. 10, 2020, which is assigned to the assignee of the present application and incorporated herein by reference.

### FIELD OF THE INVENTION

The present invention relates generally to prosthetic cardiac valves and transcatheter delivery techniques.

### BACKGROUND OF THE INVENTION

Cardiac valve diseases are the most frequent structural heart diseases. There are 500,000 annual diagnoses of severe mitral regurgitation in the US and Europe, of which only about 20% are treated surgically. The transcatheter mitral valve replacement devices currently available have several limitations, linked to the design and technology of the prosthetic frames utilized, which require crimping within a delivery shaft prior to the implantation. Conventional radial compression requires delivery systems with large diameters, and this results in higher complication rates in morbidity and mortality caused by bleeding at the access site, residual atrial septal defects, or deployment challenges.

PCT Publication WO 2019/003221 to Carmi et al. describes an intraluminal support structure having a delivery configuration that is a crimped open configuration to increase flexibility while maneuvering in the anatomy and having a small scarring signature.

### SUMMARY OF THE INVENTION

Embodiments of the present invention provide split-frame cardiac valves that are configured to be delivered to a native cardiac valve annulus in a transcatheter procedure while disposed in a delivery catheter in non-tubular radially compressed configurations.

The non-tubular configurations reduce the crossing profile of the valve compared to conventional prosthetic valves, thereby allowing the use of a lower-diameter delivery catheter, which may result in less bleeding at the access site and may provide greater controllability during deployment with a beating heart. For procedures requiring trans septal delivery, the use of a lower-diameter catheter may cause less iatrogenic damage to the atrial septum. These configurations also reduce the stress on prosthetic leaflets of the valve when they are crimped within the delivery catheter. These configurations may also allow adjustment and tailoring of the device size to match the native anatomy. In addition, these configurations may improve manufacturing processes for the prosthetic implants, simplifying and accelerating the frame and leaflet suturing processing; for example, it is generally easier to produce and post-process a circumferential segment of frame than a closed stent frame (for example, a circumferential segment can be produced from a sheet of material rather than from a laser cut tube, which is more complex and more expensive), and it generally easier to suture a single leaflet on a partial frame, accelerating the suturing process and standardizing leaflet apposition, without having to consider the apposition or interference of the other leaflets during this process.

In some applications of the present invention, the split-frame cardiac valve comprises two or more circumferential frame segments, such as exactly two or exactly three circumferential frame segments. The circumferential frame segments comprise respective stents and respective prosthetic leaflets, which are coupled to respective stents. The split-frame cardiac valve is configured such that when the split-frame cardiac valve is in a radially expanded configuration: each of the stents surrounds less than 360 degrees of a central longitudinal axis of the split-frame cardiac valve, and the circumferential frame segments are slidingly coupled to one another so as to be axially slidable with respect to one another in and out of axial alignment with one another.

The split-frame cardiac valve is configured such that when the split-frame cardiac valve is in the radially expanded configuration and the circumferential frame segments are axially aligned with one another, the stents collectively define a tubular stent that entirely surrounds the central longitudinal axis, and is configured to be anchored to the native cardiac valve annulus.

For some applications, each of the circumferential frame segments comprises two tracks disposed on opposite axially-oriented edges of the stents of respective circumferential frame segments. The two tracks of one of the circumferential frame segments axially slide with respect to the two tracks of another of the circumferential frame segments as the circumferential frame segments axially slide with respect to one another in and out of axial alignment with one another. The split-frame cardiac valve typically comprises sliders, which slidingly couple together pairs of the tracks of the two different circumferential frames.

In some other applications of the present invention, the split-frame cardiac valve comprises an axially-split tubular stent having two free longitudinal edges and two end edges, and two or more prosthetic leaflets, which are coupled to the tubular stent. The split-frame cardiac valve is configured such that when the split-frame cardiac valve is in a radially expanded configuration upon delivery from a delivery catheter and the two free axially-oriented edges are coupled together, the axially-split tubular stent assumes a tubular shape in which the longitudinal edges are axially-oriented and the two end edges are disposed at respective axial ends of the tubular stent, and the two or more prosthetic leaflets are configured to assume open and closed states.

There is therefore provided, in accordance with an application of the present invention, a split-frame cardiac valve, which is configured to assume a radially compressed configuration for transcatheter delivery and a radially expanded configuration for anchoring to a native cardiac valve annulus, the split-frame cardiac valve including:
two or more circumferential frame segments, which include:
respective stents; and
respective prosthetic leaflets, which are coupled to the respective stents, wherein the split-frame cardiac valve is configured such that when the split-frame cardiac valve is in the radially expanded configuration:
each of the stents surrounds less than 360 degrees of a central longitudinal axis of the split-frame cardiac valve, the circumferential frame segments are slidingly coupled to one another so
as to be axially slidable with respect to one another in and out of axial alignment with one another, and when the circumferential frame segments are axially aligned with one another:
the stents collectively define a tubular stent that entirely surrounds the central longitudinal axis, and is configured to be anchored to the native cardiac valve annulus, and the prosthetic leaflets are configured to assume open and closed states, and to coapt with one another when in the closed states.

For some applications, the split-frame cardiac valve includes three or more of the circumferential frame segments.

For some applications, the split-frame cardiac valve is configured such that when the split-frame cardiac valve is in the radially expanded configuration, each of the stents surrounds a same number of degrees of the central longitudinal axis.

For some applications, the split-frame cardiac valve is configured such that when the split-frame cardiac valve is in the radially expanded configuration, the stents surround respective different numbers of degrees of the central longitudinal axis.

For some applications, the split-frame cardiac valve is configured such that when the split-frame cardiac valve is in the radially expanded configuration, the tubular stent collectively defined by the stents is cylindrical.

For some applications, the split-frame cardiac valve is configured such that when the split-frame cardiac valve is in the radially expanded configuration, the tubular stent collectively defined by the stents is circularly cylindrical.

For some applications, the circumferential frame segments include tracks that slidingly couple the circumferential frame segments to one another when the split-frame cardiac valve is in the radially expanded configuration.

For some applications, the split-frame cardiac valve is configured such that when the split-frame cardiac valve is in the radially expanded configuration, the tracks are oriented parallel to the central longitudinal axis.

For some applications, the tracks are fixed to, and cantilevered with respect to, respective axially-oriented edges of the stents.

For some applications, the split-frame cardiac valve includes sliders, which are slidingly coupled to the tracks, respectively, when the split-frame cardiac valve is in the radially expanded configuration.

For some applications, the sliders are shaped so as to define respective channels that entirely surround the tracks to which the sliders are respectively slidingly coupled.

For some applications, the sliders are configured to become axially locked with respect to the tracks when the circumferential frame segments are axially aligned with one another.

For some applications, each of the circumferential frame segments includes two of the tracks, which, when the split-frame cardiac valve is in the radially expanded configuration, are disposed on opposite axially-oriented edges of the stent of the circumferential frame segment.

For some applications, the split-frame cardiac valve includes sliders, which slidingly couple together pairs of the tracks of two different circumferential frames.

For some applications, at least two of the sliders couple together each of the pairs of the tracks.

For some applications, for each of the pairs of the tracks:
a first one of the at least two sliders is axially fixed to a first one of the tracks of the pair, and axially slidable with a second one of the tracks of the pair, and
a second one of the at least two sliders is axially fixed to the second one of the tracks of the pair, and axially slidable with the first one of the tracks of the pair.

For some applications, each of the sliders that is axially slidable with one of the tracks is shaped so as to define a channel that entirely surrounds the one of the tracks.

For some applications, a number of the circumferential frame segments equals a number of the tracks.

For some applications, each of the circumferential frame segments includes one of the tracks, which, when the split-frame cardiac valve is in the radially expanded configuration, is disposed on an axially-oriented edge of the stent of the circumferential frame segment.

For some applications:
the split-frame cardiac valve includes sliders, each of the sliders is coupled to an axially-oriented edge of the stent of one of the circumferential frame segments, opposite the axially-oriented edge to which the track of the circumferential frame segment is disposed, and
each of the sliders is slidingly coupled to one of the tracks of one of the circumferential frame segments to which the slider is not coupled.

For some applications:
the split-frame cardiac valve includes two circumferential frame segments, and one of the circumferential frame segments includes two of the tracks, which, when the split-frame cardiac valve is in the radially expanded configuration, are disposed on opposite axially-oriented edges of the stent of the circumferential frame segment.

For some applications:
one of the circumferential frame segments includes two sliders, which, when the split-frame cardiac valve is in the radially expanded configuration, are disposed on opposite axially-oriented edges of the stent of the circumferential frame segment, and each of the sliders is slidingly coupled to one of the tracks.

For some applications, the cardiac valve system further including a delivery catheter in which the split-frame cardiac valve is removably disposed in the radially compressed configuration such that the two or more circumferential frame segments are at least partially non-axially-overlapping with one another.

For some applications, the split-frame cardiac valve is removably disposed in the delivery catheter in the radially compressed configuration such that the stents are entirely non-axially-overlapping with one another.

There is further provided, in accordance with an application of the present invention, a method including:
advancing a delivery catheter toward a native cardiac valve annulus in a transcatheter procedure while a split-frame cardiac valve is removably disposed in the delivery catheter in a radially compressed configuration such that two or more circumferential frame segments of the split-frame cardiac valve are at least partially non-axially-overlapping with one another, the two or more circumferential frame segments including respective stents and respective prosthetic leaflets, which are coupled to the respective stents; and anchoring the stents to the native cardiac valve annulus by:
sequentially deploying the two or more circumferential frame segments from the delivery catheter, such that the split-frame cardiac valve assumes a radially expanded configuration in which each of the stents surrounds less than 360 degrees of a central longitudinal axis of the split-frame cardiac valve, and the circumferential frame segments are slidingly coupled to one another so as to be axially slidable with respect to one another in and out of axial alignment with one another, and axially aligning the circumferential frame segments with one another outside the delivery catheter, such that:
the stents collectively define a tubular stent that entirely surrounds the central longitudinal axis, and the prosthetic leaflets are configured to assume open and closed states, and to coapt with one another when in the closed states.

For some applications, the split-frame cardiac valve includes three or more of the circumferential frame segments.

For some applications, advancing includes advancing the delivery catheter toward the native cardiac valve annulus while the split-frame cardiac valve is removably disposed in the delivery catheter in the radially compressed configuration such that the stents are entirely non-axially-overlapping with one another.

For some applications:
the circumferential frame segments include tracks that slidingly couple the circumferential frame segments to one another when the split-frame cardiac valve is in the radially expanded configuration, and axially aligning includes axially aligning the circumferential frame segments with one another outside the delivery catheter by sliding the tracks with respect to one another.

For some applications, the split-frame cardiac valve is configured such that when the split-frame cardiac valve is in the radially expanded configuration, the tracks are oriented parallel to the central longitudinal axis.

For some applications, the tracks are fixed to, and cantilevered with respect to, respective axially-oriented edges of the stents.

For some applications, the split-frame cardiac valve includes sliders, which are slidingly coupled to the tracks, respectively, when the split-frame cardiac valve is in the radially expanded configuration.

For some applications, the sliders are shaped so as to define respective channels that entirely surround the tracks to which the sliders are respectively slidingly coupled.

For some applications, axially aligning the circumferential frame segments with one another includes axially locking the sliders with respect to the tracks after axially aligning the circumferential frame segments with one another.

For some applications, each of the circumferential frame segments includes two of the tracks, which, when the split-frame cardiac valve is in the radially expanded configuration, are disposed on opposite axially-oriented edges of the stent of the circumferential frame segment.

For some applications:
the split-frame cardiac valve includes sliders, which slidingly couple together pairs of the tracks of two different circumferential frames, and axially aligning includes axially aligning the circumferential frame segments with one another outside the delivery catheter by sliding the pairs of the tracks with respect to one another.

For some applications, at least two of the sliders couple together each of the pairs of the tracks.

For some applications, for each of the pairs of the tracks:
a first one of the at least two sliders is axially fixed to a first one of the tracks of the pair, and axially slidable with a second one of the tracks of the pair, and a second one of the at least two sliders is axially fixed to the second one of the tracks of the pair, and axially slidable with the first one of the tracks of the pair.

For some applications, each of the sliders that is axially slidable with one of the tracks is shaped so as to define a channel that entirely surrounds the one of the tracks.

For some applications, a number of the circumferential frame segments equals a number of the tracks.

For some applications, each of the circumferential frame segments includes one of the tracks, which, when the split-frame cardiac valve is in the radially expanded configuration, is disposed on an axially-oriented edge of the stent of the circumferential frame segment.

For some applications:
the split-frame cardiac valve includes sliders, each of the sliders is coupled to an axially-oriented edge of the stent of one of the circumferential frame segments, opposite the axially-oriented edge to which the track of the circumferential frame segment is disposed, and
each of the sliders is slidingly coupled to one of the tracks of one of the circumferential frame segments to which the slider is not coupled.

For some applications:
the split-frame cardiac valve includes two circumferential frame segments, and one of the circumferential frame segments includes two of the tracks, which, when the split-frame cardiac valve is in the radially expanded configuration, are disposed on opposite axially-oriented edges of the stent of the circumferential frame segment.

For some applications:
one of the circumferential frame segments includes two sliders, which, when the split-frame cardiac valve is in the radially expanded configuration, are disposed on opposite axially-oriented edges of the stent of the circumferential frame segment, and each of the sliders is slidingly coupled to one of the tracks.

There is still further provided, in accordance with an application of the present invention, a split-frame cardiac valve, which is configured to assume a radially compressed configuration for transcatheter delivery and a radially expanded configuration for anchoring to a native cardiac valve annulus, the split-frame cardiac valve including:
two or more circumferential frame segments, which include respective first stents, wherein one of the first stents includes one or more first couplers;
a leaflet assembly, which includes a second stent and one or more second couplers; and
two or more prosthetic leaflets, wherein one of the prosthetic leaflets is coupled to the second stent of the leaflet assembly, and the remaining one or more prosthetic leaflets are coupled to one or more of the first stents, respectively, such that the one of first stents that includes the one or more first couplers is not coupled to any of the prosthetic leaflets, wherein the split-frame cardiac valve is configured such that when the split-frame cardiac valve is in the radially expanded configuration:
(i) each of the first stents surrounds less than 360 degrees of a central longitudinal axis of the split-frame cardiac valve,
(ii) the circumferential frame segments are slidingly coupled to one another so as to be axially slidable with respect to one another in and out of axial alignment with one another,
(iii) when the circumferential frame segments are axially aligned with one another:
   the first stents collectively define a tubular stent that entirely surrounds the central longitudinal axis, and is configured to be anchored to the native cardiac valve annulus, and
   the two or more prosthetic leaflets are configured to assume open and closed states, and to coapt with one another when in the closed states, and
(iv) the leaflet assembly is couplable to the one of the first stents that is not coupled to any of the prosthetic leaflets, by coupling the one or more second couplers to the one or more first couplers, respectively.

For some applications, the split-frame cardiac valve includes three or more of the circumferential frame segments and three or more of the prosthetic leaflets.

There is additionally provided, in accordance with an application of the present invention, a method including:
advancing a delivery catheter toward a native cardiac valve annulus in a transcatheter procedure while a split-frame cardiac valve is removably disposed in the delivery catheter in a radially compressed configuration such that two or more circumferential frame segments of the split-frame cardiac valve are at least partially non-axially-overlapping with one another, the two or more circumferential frame segments including respective first stents, wherein one of the first stents includes one or more first couplers, wherein the split-frame cardiac valve further includes (a) a leaflet assembly, which includes a second stent and one or more second couplers, and (b) two or more prosthetic leaflets, wherein one of the prosthetic leaflets is coupled to the second stent of the leaflet assembly, and the remaining one or more prosthetic leaflets are coupled to one or more of the first stents, respectively, such that the one of first stents that includes the one or more first couplers is not coupled to any of the prosthetic leaflets,
anchoring the first stents to the native cardiac valve annulus by:
sequentially deploying the two or more circumferential frame segments from the delivery catheter, such that the split-frame cardiac valve assumes a radially expanded configuration in which each of the first stents surrounds less than 360 degrees of a central longitudinal axis of the split-frame cardiac valve, and the circumferential frame segments are slidingly coupled to one another so as to be axially slidable with respect to one another in and out of axial alignment with one another, and
axially aligning the circumferential frame segments with one another outside the delivery catheter, such that:
the first stents collectively define a tubular stent that entirely surrounds the central longitudinal axis, and
the two or more prosthetic leaflets are configured to assume open and closed states, and to coapt with one another when in the closed states; before or after axially aligning the circumferential frame segments with one another, coupling the leaflet assembly to the one of the first stents that is not coupled to any of the prosthetic leaflets, by coupling the one or more second couplers to the one or more first couplers, respectively.

For some applications, the split-frame cardiac valve includes three or more of the circumferential frame segments and three or more of the prosthetic leaflets.

There is yet additionally provided, in accordance with an application of the present invention, a cardiac valve system including:
a split-frame cardiac valve, which includes:
an axially-split tubular stent having two free longitudinal edges and two end edges; and
two or more prosthetic leaflets, which are coupled to the tubular stent; and
a delivery catheter in which the split-frame cardiac valve is removably disposed with the axially-split tubular stent radially compressed in a three-dimensional spiral configuration, with one of the free longitudinal edges disposed distal-most in the delivery catheter and the other of the free longitudinal edges disposed proximal-most in the delivery catheter,
wherein the split-frame cardiac valve is configured such that when the split-frame cardiac valve is in a radially expanded configuration upon delivery from the delivery catheter and the two free longitudinal edges are coupled together, the axially-split tubular stent assumes a tubular shape in which:
the two free longitudinal edges are axially-oriented and the two end edges are disposed at respective axial ends of the tubular stent, and the prosthetic leaflets are configured to assume open and closed states, and
to coapt with one another when in the closed states.

There is also provided, in accordance with an application of the present invention, a method including:
advancing a delivery catheter toward a native cardiac valve annulus in a transcatheter procedure while a split-frame cardiac valve is removably disposed in the delivery catheter with an axially-split tubular stent of the split-frame cardiac valve radially compressed in a three-dimensional spiral configuration, with one of two free longitudinal edges of the axially-split tubular stent disposed distal-most in the delivery catheter and the other of the two free longitudinal edges disposed proximal-most in the delivery catheter,
wherein the split-frame cardiac valve further includes two or more prosthetic leaflets, which are coupled to the tubular stent;
deploying the split-frame cardiac valve from the delivery catheter, such that the split-frame cardiac valve assumes a radially expanded configuration; and
coupling together the two free axially-oriented edges such that the axially-split tubular stent assumes a tubular shape in which the two free longitudinal edges are axially-oriented and two end edges of the axially-split tubular stent are disposed at respective axial ends of the tubular stent, and the prosthetic leaflets are configured to assume open and closed states, and to coapt with one another when in the closed states.

There is further provided, in accordance with an application of the present invention, a cardiac valve system including:
a split-frame cardiac valve, which includes:
an axially-split tubular stent having first and second free longitudinal edges and proximal and distal end edges; and
two or more prosthetic leaflets, which are coupled to the tubular stent; and a delivery catheter in which the split-frame cardiac valve is removably disposed with the axially-split tubular stent radially compressed in a rolled configuration defining more than one turn, in which the first and the second free longitudinal edges are axially-oriented parallel to each other and to a central longitudinal axis of the delivery catheter, and the first free longitudinal edge is disposed more distally than the second free longitudinal edge, wherein the split-frame cardiac valve is configured such that when the split-frame cardiac valve is in a radially expanded configuration upon deployment from a distal end of the delivery catheter and the first and the second free longitudinal edges are coupled together, the axially-split tubular stent assumes a tubular shape in which: the first and the second free longitudinal edges are axially-oriented and the proximal and the distal end edges are disposed at respective axial ends of the tubular stent, and
the prosthetic leaflets are configured to assume open and closed states, and
to coapt with one another when in the closed states.

For some applications, the split-frame cardiac valve is removably disposed within the delivery catheter with the axially-split tubular stent radially compressed in the rolled configuration in which a proximal end of the first free longitudinal edge is disposed more distally than a distal end of the second free longitudinal edge.

For some applications, the split-frame cardiac valve is removably disposed within the delivery catheter with the axially-split tubular stent radially compressed in the rolled configuration in which a distance between the proximal end of the first free longitudinal edge and the distal end of the second free longitudinal edge equals at least 25% of a length of the first free longitudinal edge.

For some applications, the split-frame cardiac valve further includes:
a shaft, which is coupled to the first free longitudinal edge; and
a shaft receptacle, which is coupled to the second free longitudinal edge, and is configured to slidingly accept the shaft, such that upon insertion of the shaft into the shaft receptacle after deployment of the split-frame cardiac valve from the distal end of the delivery catheter, the first and the second free longitudinal edges are coupled together by the shaft and the shaft receptacle.

For some applications, the apparatus further includes a deployment wire, which is removably coupled to a proximal end of the shaft and passes through the shaft receptacle and the delivery catheter when the split-frame cardiac valve is removably disposed within the delivery catheter.

For some applications, the shaft receptacle is at least partially cylindrical.

For some applications, the shaft receptacle is shaped as a partial cylinder that defines a longitudinal slot therealong.

For some applications, the shaft is shaped so as to define one or more couplers that are configured to engage the shaft receptacle to prevent decoupling of the shaft from the shaft receptacle after insertion therein.

For some applications, a distal end of the shaft includes a stopper, which limits advancement of the shaft into the shaft receptacle.

There is still further provided, in accordance with an application of the present invention, a method including:
advancing a delivery catheter toward a native cardiac valve annulus in a transcatheter procedure while a split-frame cardiac valve is removably disposed in the delivery catheter with an axially-split tubular stent of the split-frame cardiac valve radially compressed in a rolled configuration defining more than one turn, in which first and second free longitudinal edges of the tubular stent axially-oriented parallel to each other and to a central longitudinal axis of the delivery catheter, and a first free longitudinal edge of the tubular stent is disposed more distally than a second free longitudinal edge of the tubular stent, wherein the split-frame cardiac valve further includes two or more prosthetic leaflets, which are coupled to the tubular stent;
deploying the split-frame cardiac valve from a distal end of the delivery catheter, such that the split-frame cardiac valve assumes a radially expanded configuration; and coupling together the first and the second free longitudinal edges such that the axially-split tubular stent assumes a tubular shape in which the first and the second free longitudinal edges are axially-oriented and proximal and distal end edges of the tubular stent are disposed at respective axial ends of the tubular stent, and the prosthetic leaflets are configured to assume open and closed states, and to coapt with one another when in the closed states.

For some applications, advancing the delivery catheter includes advancing the delivery catheter while the split-frame cardiac valve is removably disposed within the delivery catheter with the axially-split tubular stent radially compressed in the rolled configuration in which a proximal end of the first free longitudinal edge is disposed more distally than a distal end of the second free longitudinal edge.

For some applications, advancing the delivery catheter includes advancing the delivery catheter while the split-frame cardiac valve is removably disposed within the delivery catheter with the axially-split tubular stent radially compressed in the rolled configuration in which a distance between the proximal end of the first free longitudinal edge and the distal end of the second free longitudinal edge equals at least 25% of a length of the first free longitudinal edge.

For some applications:
the split-frame cardiac valve further includes (a) a shaft, which is coupled to the first free longitudinal edge, and (b) a shaft receptacle, which is coupled to the second free longitudinal edge, and coupling together the first and the second free longitudinal edges includes sliding the shaft into the shaft receptacle such that the first and the second free longitudinal edges are coupled together by the shaft and the shaft receptacle.

For some applications, coupling together the first and the second free longitudinal edges further includes, after sliding the shaft into the shaft receptacle, further coupling together the first and the second free longitudinal edges using one or more coupling elements selected from the group consisting of: one or more sutures and one or more anchors.

For some applications, sliding the shaft into the shaft receptacle includes pulling, in a proximal direction, a deployment wire that is removably coupled to a proximal end of the shaft and passes through the shaft receptacle and the delivery catheter when the split-frame cardiac valve is removably disposed within the delivery catheter.

For some applications, the shaft receptacle is at least partially cylindrical.

For some applications, the shaft receptacle is shaped as a partial cylinder that defines a longitudinal slot therealong.

For some applications, the shaft is shaped so as to define one or more couplers that are configured to engage the shaft receptacle to prevent decoupling of the shaft from the shaft receptacle after insertion therein.

For some applications, a distal end of the shaft includes a stopper, which limits advancement of the shaft into the shaft receptacle.

For some applications, coupling together the first and the second free longitudinal edges includes coupling together the first and the second free longitudinal edges using one or more coupling elements selected from the group consisting of: one or more sutures and one or more anchors.

The present invention will be more fully understood from the following detailed description of embodiments thereof, taken together with the drawings, in which:

### BRIEF DESCRIPTION OF THE DRAWINGS

FIGS. 1A-B are schematic illustrations of a split-frame cardiac valve comprising two circumferential frame segments, in accordance with an application of the present invention;

FIGS. 2A-C are additional schematic illustrations of the split-frame cardiac valve of FIGS. 1A-B, in accordance with an application of the present invention;

FIG. 3 is yet an additional schematic illustration of the split-frame cardiac valve of FIGS. 1A-B, in accordance with an application of the present invention;

FIGS. 4A-B are further schematic illustrations of the split-frame cardiac valve of FIGS. 1A-B, in accordance with an application of the present invention;

FIGS. 5A-C are schematic illustrations of another configuration of the split-frame cardiac valve of FIGS. 1A-B, in accordance with an application of the present invention;

FIGS. 6A and 6B are schematic illustrations of additional configurations of the split-frame cardiac valve of FIGS. 1A-B, in accordance with respective applications of the present invention;

FIGS. 7A-B are schematic illustrations of a split-frame cardiac valve comprising three circumferential frame segments, in accordance with an application of the present invention;

FIGS. 8A-B are additional schematic illustrations of the split-frame cardiac valve of FIGS. 7A-B, in accordance with an application of the present invention;

FIGS. 8C-D are schematic illustrations of another configuration of the split-frame cardiac valve of FIGS. 7A-B, in accordance with an application of the present invention;

FIG. 9 is a schematic illustration of a cardiac valve system that comprises the split-frame cardiac valve of FIGS. 1A-B and a delivery catheter, in accordance with an application of the present invention;

FIGS. 10A-B are schematic illustrations of another split-frame cardiac valve, in accordance with an application of the present invention;

FIGS. 11A-C are schematic illustrations of the split-frame cardiac valve of FIGS. 10A-B after two free longitudinal edges of an axially-split tubular stent thereof have been coupled together, in accordance with respective applications of the present invention;

FIGS. 12A-C are schematic illustrations of a method for deploying the split-frame cardiac valve of FIGS. 10A-B in a native cardiac valve annulus, in accordance with an application of the present invention; and

FIGS. 13A-C are schematic illustrations of another split-frame cardiac valve and a method of deployment, in accordance with an application of the present invention.

### DETAILED DESCRIPTION OF EMBODIMENTS

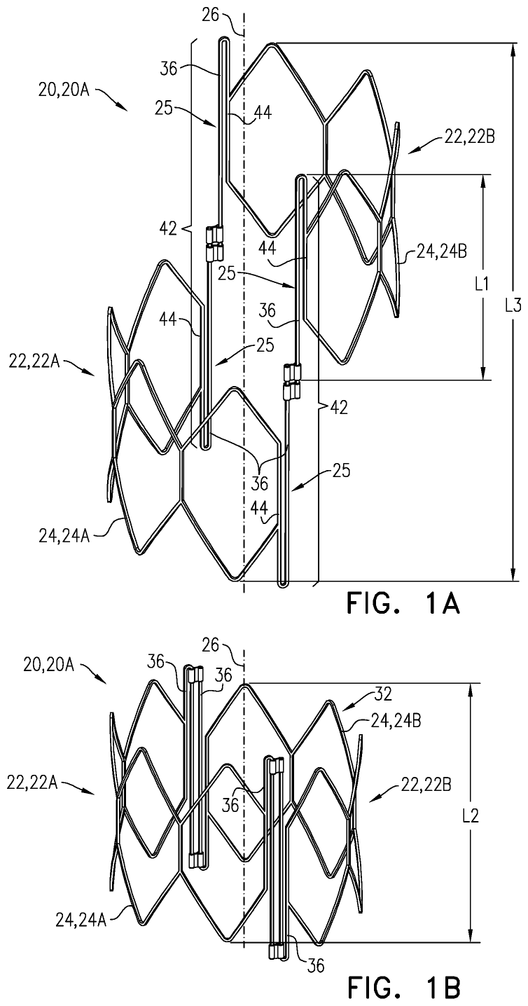

Reference is made to FIGS. 1A-B, 2A-C, 3, and 4A-B, which are schematic illustrations of a split-frame cardiac valve 20, 20A, in accordance with an application of the present invention.

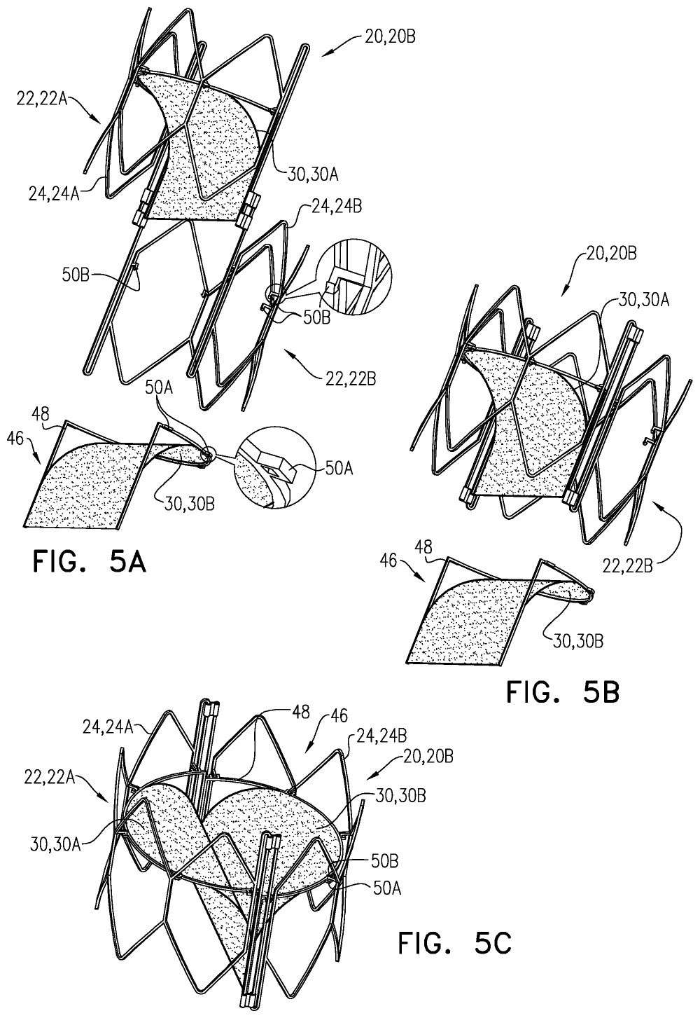

Reference is further made to FIGS. 5A-C, which are schematic illustrations of a split-frame cardiac valve 20, 20B, in accordance with an application of the present invention.

(Although skirt portions 34 are not shown in FIGS. 5A-C, they may optionally be provided.)

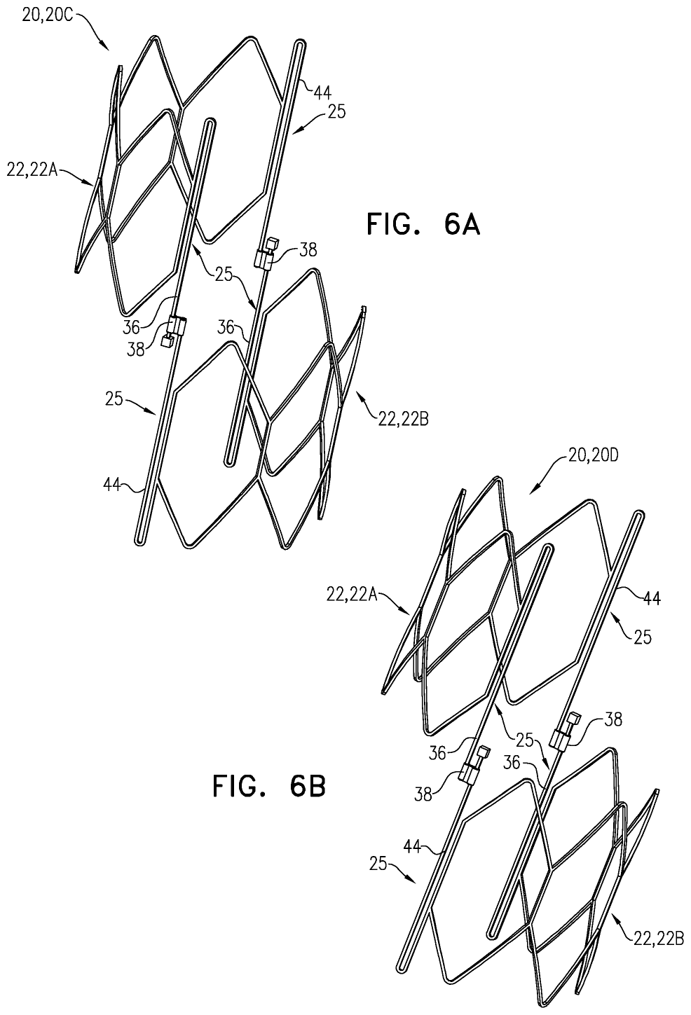

Reference is still further made to FIGS. 6A and 6B, which are schematic illustrations of a split-frame cardiac valve 20, 20C and a split-frame cardiac valve 20, 20E, respectively, in accordance with respective applications of the present invention.

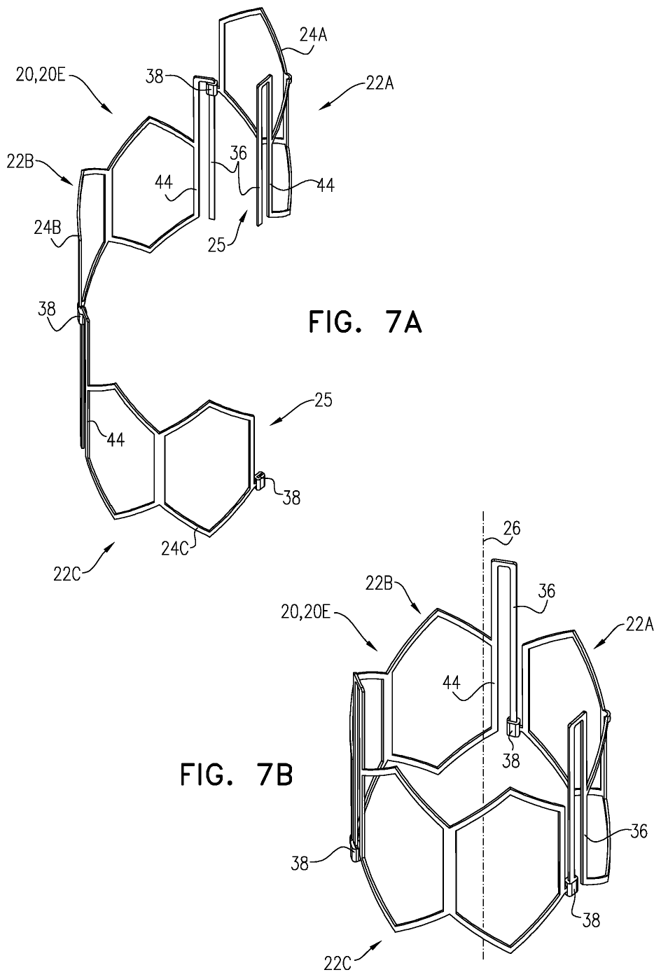

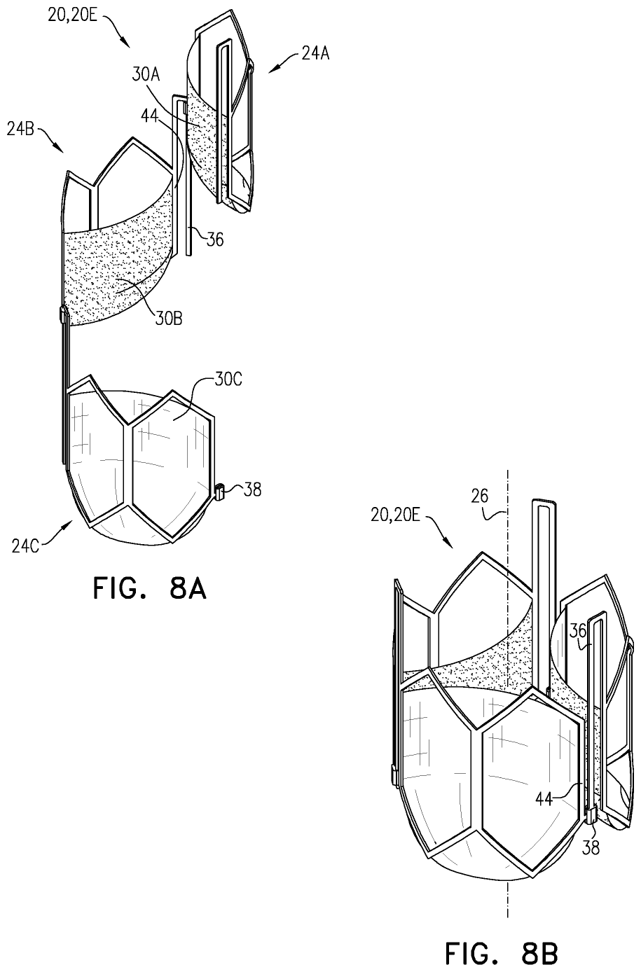

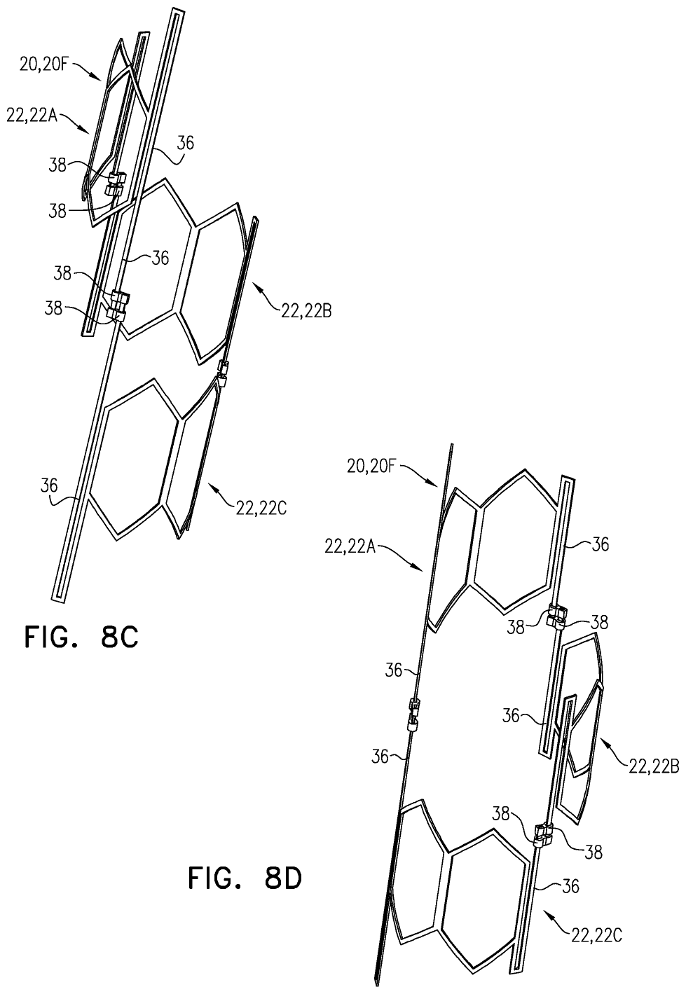

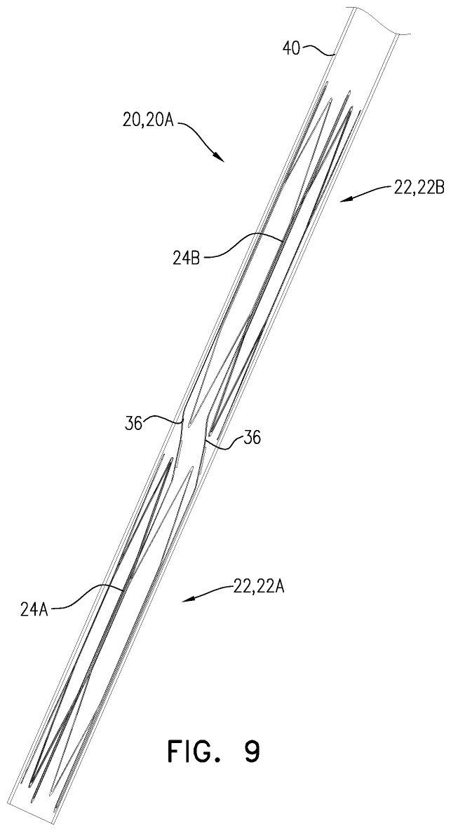

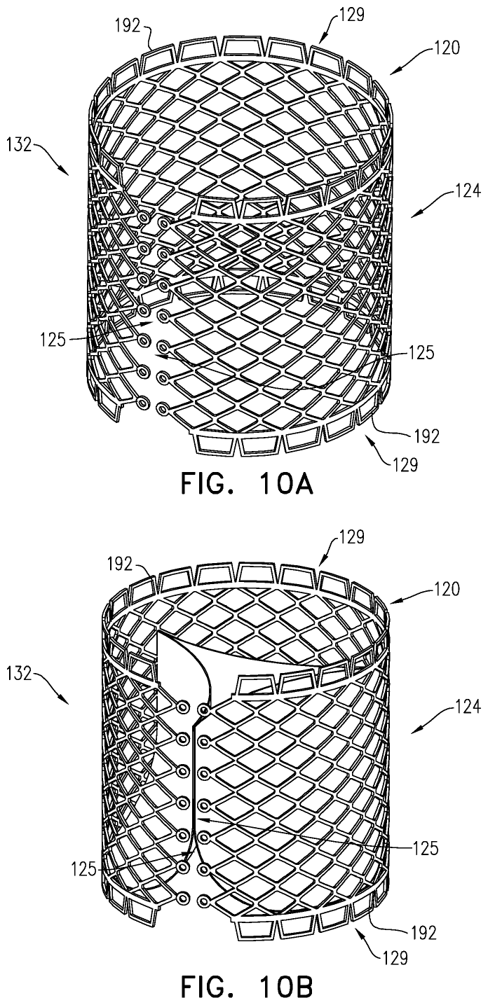

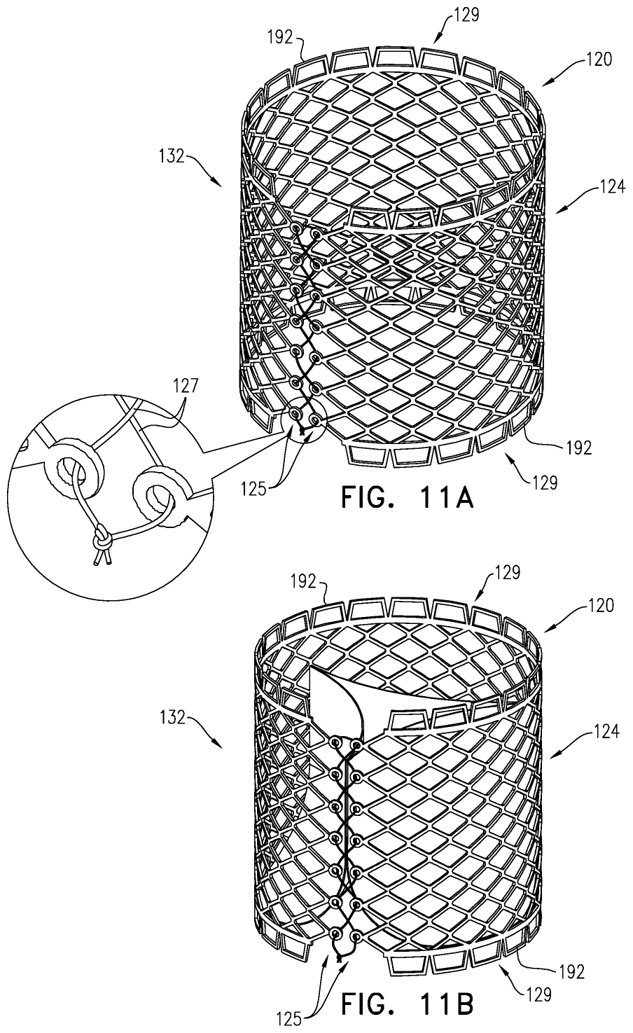

Split-frame cardiac valve 20 is configured to be implanted in a transcatheter procedure to repair or replace a native cardiac valve. For example, the transcatheter procedure may be a transapical procedure or a transvascular procedure. Split-frame cardiac valve 20 is configured to assume a radially compressed configuration for transcatheter delivery, such as described hereinbelow with reference to FIG. 9, and a radially expanded configuration, such as shown in FIGS. 1A-6B (and FIGS. 7A-8B), for anchoring to a native cardiac valve annulus.

Split-frame cardiac valve 20 comprises two or more circumferential frame segments 22, such as (a) exactly two circumferential frame segments 22A and 22B, such as shown in FIGS. 1A-B, 2A-C, 3, and 4A-B for split-frame cardiac valve 20A, in FIGS. 5A-C for split-frame cardiac valve 20A, 20B, and in FIGS. 6A and 6B for split-frame cardiac valves 20C and 20D, respectively; (b) exactly three circumferential frame segments 22A, 22B, and 22C, such as shown and described hereinbelow with reference to FIGS. 7A-B and 8A-B for a split-frame cardiac valve 20E and with reference to FIGS. 8C-D for a split-frame cardiac valve 20F; or (c) more than three circumferential frame segments, such as four or five circumferential frame segments (configuration not shown).

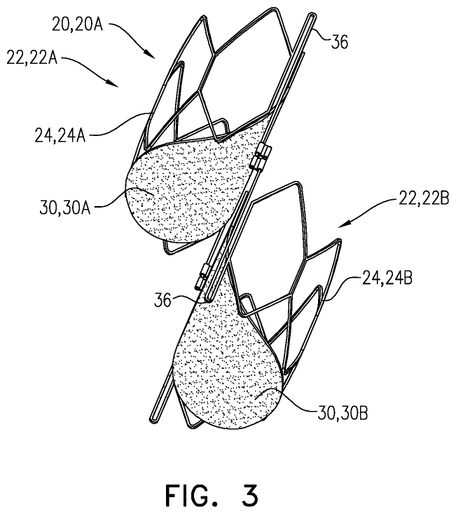

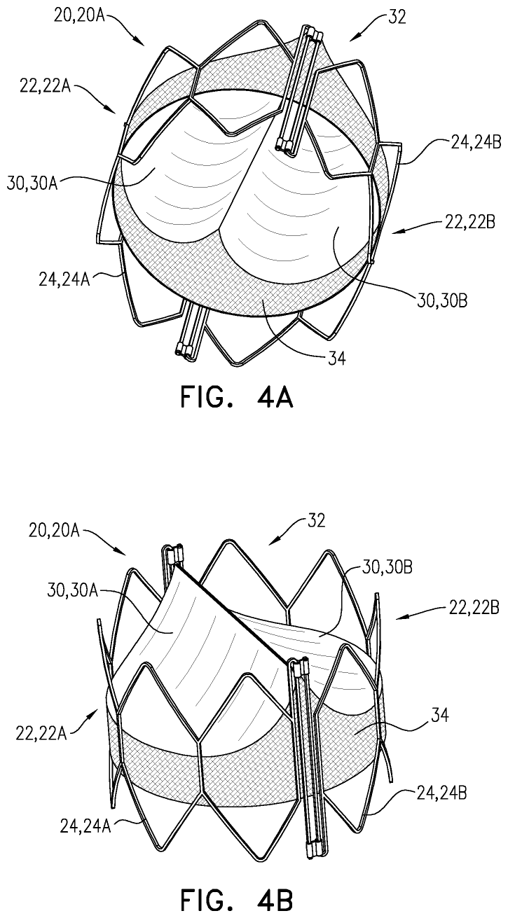

For some applications, circumferential frame segments 22A and 22B comprise:
respective stents 24A and 24B, which typically comprise interconnected struts, which may be arranged in any pattern known in the stent art, such as in cells (e.g., diamond cells), in bands, e.g., serpentine or sinusoidal bands, or a combination of cells and bands; and
respective prosthetic leaflets 30A and 30B, which are coupled to respective stents 24A and 24B (prosthetic leaflets 30 are shown in FIGS. 3, 4A-B, and 5A-C, and, for clarity of illustration, are not shown in FIGS. 1A-B, 2A-C, 6A-B, 7A-B, 8A-B, and 9).

Split-frame cardiac valve 20 is configured such that when split-frame cardiac valve 20 is in the radially expanded configuration:
each of stents 24A and 24B surrounds less than 360 degrees of a central longitudinal axis 26 of split-frame cardiac valve 20, and
circumferential frame segments 22A and 22B are slidingly coupled to one another so as to be axially slidable with respect to one another in and out of axial alignment with one another.

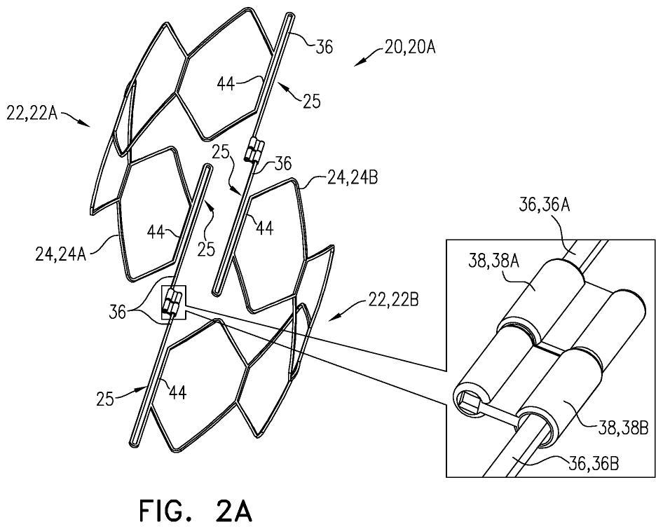

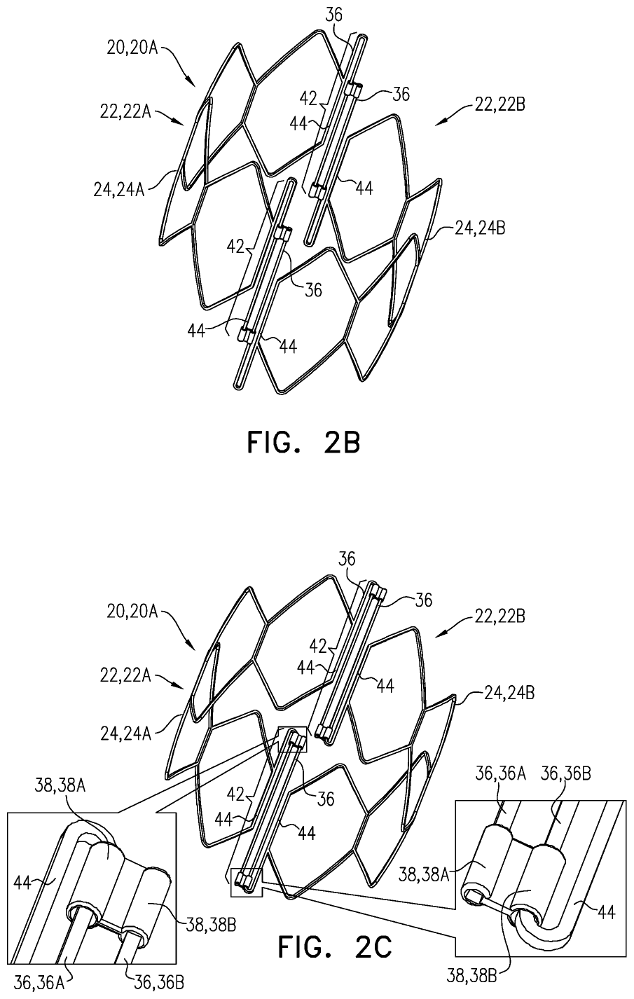

FIGS. 1A, 2A, 3, 5A, 6A-B, 7A, 8A, 8C-D, and 9 show split-frame cardiac valve 20, 20A, 20B, 20C, 20D, 20E, 20F with circumferential frame segments 22A and 22B out of axial alignment with one another, and FIGS. 1B, 2C, 4A-B, 5B-C, 7B, and 8B show split-frame cardiac valve 20, 20A, 20B, 20E with circumferential frame segments 22A and 22B in axial alignment with one another. FIG. 2B shows split-frame cardiac valve 20, 20A with circumferential frame segments 22A and 22B partially axially aligned with one another during a transition between the alignments shown in FIG. 2A and FIG. 2C.

FIGS. 3 and 4A-B show configurations in which both circumferential frame segments 22A and 22B comprise respective prosthetic leaflets 30A and 30B.

Optionally, one or more of circumferential frame segments 22 comprise respective skirt portions 34, such as shown in FIGS. 4A-B.

As shown in FIGS. 1B, 2B, 4A-B, 5B-C, and 5B-C, split-frame cardiac valve 20 is configured such that when split-frame cardiac valve 20 is in the radially expanded configuration and circumferential frame segments 22A and 22B are axially aligned with one another, stents 24A and 24B collectively define a tubular stent 32 that entirely surrounds central longitudinal axis 26 (i.e., surround 360 degrees of central longitudinal axis 26), and is configured to be anchored to the native cardiac valve annulus. In addition, as shown in FIGS. 4A-B, in configurations in which both circumferential frame segments 22A and 22B comprise respective prosthetic leaflets 30A and 30B, split-frame cardiac valve 20 is configured such that when circumferential frame segments 22A and 22B are axially aligned with one another, prosthetic leaflets 30A and 30B are configured to assume open and closed states, and to coapt with one another when in the closed states. (The circumferential frame segments 22 are referred to as “circumferential” because frame segments 22 (as well as their respective stents 24) extend around only a portion of the circumference of tubular stent 32 when frame segments 22 are axially aligned with one another.)

For some applications, each of stents 24A and 24B surrounds a same number of degrees of central longitudinal axis 26 (as shown in the figures), while for other applications, stents 24A and 24B surround respective different numbers of degrees of central longitudinal axis 26 (configuration not shown).

For some applications, tubular stent 32 collectively defined by stents 24A and 24B is cylindrical, e.g., circularly cylindrical (such as shown in the figures) or non-circularly elliptically cylindrical (configuration not shown).

Reference is made to FIGS. 5A-C. In this configuration, circumferential frame segment 22A comprises a prosthetic leaflet 30A, and circumferential frame segment 22B does not comprise a prosthetic leaflet 30. Instead, split-frame cardiac valve 20, 20B comprises a separate leaflet assembly 46, which comprises prosthetic leaflet 30B and a stent 48 that is coupled to and supports prosthetic leaflet 30B. Leaflet assembly 46 and circumferential frame segment 22B comprise respective couplers 50A and 50B that are configured to be coupled together, as shown in FIG. 5C, in order to couple leaflet assembly 46 to circumferential frame segment 22B in situ after deployment from a delivery catheter, either after circumferential frame segments 22A and 22B have been axially aligned with one another, as shown in FIG. 5B, or before circumferential frame segments 22A and 22B have been axially aligned with one another (configuration not shown).

In configurations of split-frame cardiac valve 20 that comprise three or more circumferential frame segments 22, such as described hereinbelow with reference to FIGS. 7A-B, 8A-B, and 8C-D, optionally only a single one of the three or more circumferential frame segments 22 (e.g., of the exactly three segments 22) comprises a leaflet 30, or exactly two of the three or more circumferential frame segments 22 (e.g., of the exactly three segments 22) comprise respective leaflets 30. In other words, optionally at least one of the circumferential frame segments 22 does not comprise a leaflet 30, i.e., split-frame cardiac valve 20 comprises fewer prosthetic leaflets 30 than circumferential frame segments 22.

Typically, a corresponding number of separate leaflet assemblies 46 are provided to provide the leaflets 30 lacking in the circumferential frame segments. Each of the one or more separate leaflet assemblies 46 is couplable to a respective one of the circumferential frame segments.

Reference is still made to FIGS. 1A-6B. For some applications, circumferential frame segments 22 comprise tracks 36 that slidingly couple circumferential frame segments 22 to one another when split-frame cardiac valve 20 is in the radially expanded configuration. For some applications, tracks 36 are oriented parallel to central longitudinal axis 26, such as shown. Alternatively, tracks 36 are oriented at an angle with respect to central longitudinal axis 26 (configuration not shown).

Reference is made to FIGS. 1A-4B. For some applications, each of circumferential frame segments 22A and 22B of split-frame cardiac valve 20, 20A comprises two tracks 36, which, when split-frame cardiac valve 20 is in the radially expanded configuration, are disposed on opposite axially-oriented edges 25 of the stent of the circumferential frame segment. The two tracks 36 of circumferential frame segment 22A axially slide with respect to the two tracks 36 of circumferential frame segment 22B as circumferential frame segments 22A and 22B axially slide with respect to one another in and out of axial alignment with one another. (The “axially-oriented edges” are the edges of the stent that extend axially along the stent parallel to central longitudinal axis 26, and not the edges of the bases of the stent.)

Tracks 36 are typically fixed to respective axially-oriented edges 25 of stents 24A and 24B. For some applications, axially-oriented edges 25 of stents 24A and 24B are defined by respective struts 44 of stents 24A and 24B, which may or may not be parallel to tracks 36; struts 44 are not portions of tracks 36.

Reference is again made to FIGS. 1A-6B. Split-frame cardiac valve 20 typically comprises sliders 38 (labeled in FIGS. 2A-C, 6A-B, and 8C-D), which are slidingly coupled to tracks 36, respectively, when split-frame cardiac valve 20, 20A, 20B, 20C, 20D is in the radially expanded configuration. For some applications, sliders 38 are shaped so as to define respective channels that entirely surround tracks 36 to which sliders 38 are respectively slidingly coupled, such as shown. Alternatively, sliders 38 are shaped so as to define respective elongate indentations that surrounds more than 180 degrees of tracks 36 (configuration not shown).

Reference is still made to FIGS. 1A-6B. For some applications, sliders 38 are configured to become axially locked with respect to tracks 36 when circumferential frame segments 22A and 22B are axially aligned with one another. For example, the locking may be implemented using friction or a one-way slot.

Reference is still made to FIGS. 1A-6B. Typically, tracks 36 are fixed to, and cantilevered with respect to, respective axially-oriented edges 25 of stents 24 (i.e., fixed and supported at only one end of the track, while the other end is free), which allows sliding of sliders 38 along the tracks unimpeded by struts of the stents.

Reference is again made to FIGS. 1A-4B. For some applications, each of circumferential frame segments 22A and 22B of split-frame cardiac valve 20, 20A comprises two of tracks 36, which, when split-frame cardiac valve 20, 20A is in the radially expanded configuration, are disposed on opposite axially-oriented edges of the stent of the circumferential frame segment.

For some of these applications, split-frame cardiac valve 20, 20A comprises sliders 38, which slidingly couple together pairs 42 of tracks 36 of two different circumferential frames (labeled in FIGS. 1A and 2C). (Each pair 42 consists of two tracks 36A and 36B.)

The two tracks 36A and 36B of each pair 42 are parallel to each other. Typically, each of tracks 36 is cantilevered with respect to one of axially-oriented edges 25 of one of stents 24A and 24B (i.e., fixed and supported at only one end of the track, while the other end is free), which allows sliding of sliders 38 along the tracks unimpeded by struts of the stents.

Reference is still made to FIGS. 1A-4B. For some applications, at least two of sliders 38 (e.g., exactly two sliders (as shown) or exactly three sliders (configuration not shown)) couple each of pairs 42 of tracks 36 together. For example:
a first one 38A of the at least two sliders 38 may be axially fixed to a first one 36A of tracks 36 of pair 42, and axially slidable with a second one 36B of tracks 36 of pair 42, and
a second one 38B of the at least two sliders 38 may be axially fixed to the second one 36B of tracks 36 of pair 42, and axially slidable with the first one 36A of tracks 36 of pair 42.

Reference is still made to FIGS. 1A-4B. For some applications, each of sliders 38 that is axially slidable with one of tracks 36 is shaped so as to define a channel that entirely surrounds the one of tracks 36, such as shown. Alternatively, each of sliders 38 that is axially slidable with one of tracks 36 is shaped so as to define an elongate indentation that surrounds more than 180 degrees of the one of tracks 36 (configuration not shown).

Reference is made to FIGS. 6A-B. For some applications, a number of circumferential frame segments 22 equals a number of tracks 36 of split-frame cardiac valve 20, 20C, 20D; in other words, split-frame cardiac valve 20, 20C, 20D comprises the same number of circumferential frame segments 22 as tracks 36. For example, the number may be two, such as shown in FIGS. 6A-B, or three, such as shown for split-frame cardiac valve 20, 20C, 20E in FIGS. 7A-B and 8A-B, described hereinbelow.

Reference is made to FIG. 6A. For some applications, each of circumferential frame segments 22 of split-frame cardiac valve 20, 20C comprises one of tracks 36, which, when split-frame cardiac valve 20, 20C is in the radially expanded configuration, is disposed on an axially-oriented edge 25 of stent 24 of the circumferential frame segment 22.

For some of these applications in which split-frame cardiac valve 20, 20C comprises sliders 38, each of sliders 38 is coupled to an axially-oriented edge 25 of the stent of one of the circumferential frame segments 22, opposite the axially-oriented edge 25 to which tracks 22 36 of the circumferential frame segment 22 is disposed. Each of sliders 38 is slidingly coupled to one of tracks 36 of one of the circumferential frame segments 22 to which slider 38 is not coupled.

Reference is made to FIG. 6B. For some applications, one of circumferential frame segments 22 of split-frame cardiac valve 20, 20D comprises two of tracks 36, which, when split-frame cardiac valve 20, 20D is in the radially expanded configuration, are disposed on opposite axially-oriented edges 25 of stent 24 of the circumferential frame segment 22.

For some of these applications in which split-frame cardiac valve 20, 20D comprises sliders 38, one of the circumferential frame segments 22 comprises two sliders 38, which, when split-frame cardiac valve 20, 20D is in the radially expanded configuration, are disposed on opposite axially-oriented edges 25 of stent 24 of the circumferential frame segment 22. Each of sliders 38 is slidingly coupled to one of the tracks 36.

Reference is made to FIGS. 7A-B and 8A-B, which are schematic illustrations of a split-frame cardiac valve 20, 20E, in accordance with respective applications of the present invention. Reference is also made to FIGS. 8C-D, which are schematic illustrations of a split-frame cardiac valve 20, 20F, in accordance with an application of the present invention. Split-frame cardiac valve 20E, 20F may optionally implement any of the features of split-frame cardiac valve 20A, described hereinabove with reference to FIGS. 1A-6B, mutatis mutandis.

Split-frame cardiac valve 20E, 20F comprises exactly three circumferential frame segments 22A, 22B, and 22C, which, for some applications, comprise:
respective stents 24A, 24B, and 24C, (a) which are configured to be anchored to a native cardiac valve annulus, (b) each of which surrounds less than 360 degrees of a central longitudinal axis 26, and (c) which collectively entirely surround central longitudinal axis 26 (i.e., surround 360 degrees of central longitudinal axis 26); and
respective prosthetic leaflets 30A, 30B, and 30C, which are coupled to respective stents 24A, 24B, and 24C (prosthetic leaflets 30A, 30B, and 30C are shown in FIGS. 8A-B, and, for clarity of illustration, are not shown in FIGS. 7A-B).

Alternatively, at least one of the circumferential frame segments 22 does not comprise a prosthetic leaflet 30, such as described hereinabove with reference to FIG. 3A.

Circumferential frame segments 22A, 22B, and 22C are slidingly coupled to one 23 another so as to be axially slidable with respect to one another in and out of axial alignment with one another. FIGS. 7A and 8A show split-frame cardiac valve 20E with circumferential frame segments 22A, 22B, and 22C out of axial alignment with one another, and FIGS. 7B and 8B show split-frame cardiac valve 20E with circumferential frame segments 22A, 22B, and 22C in axial alignment with one another.

For some applications, each of stents 24A, 24B, and 24C surrounds a same number of degrees of central longitudinal axis 26 (as shown in the figures), while for other applications, stents 24A, 24B, and 24C surround respective different numbers of degrees of central longitudinal axis 26 (configuration not shown).

For some applications, split-frame cardiac valve 20E comprises tracks 36 and sliders 38, such as described hereinabove with reference to FIGS. 1A-6B, mutatis mutandis. For some applications, a number of circumferential frame segments 22 equals a number of tracks 36 of split-frame cardiac valve 20E, such as shown in FIGS. 7A-B and 7B, or arranged, mutatis mutandis, as shown in FIG. 6B for split-frame cardiac valve 20D. For other applications, each of circumferential frame segments 22 comprises two tracks 36, such as described hereinbelow with reference to FIGS. 8C-D.

Reference is made to FIGS. 8C-D, which are two views of split-frame cardiac valve 20, 20F in the same state (circumferential frame segments 22A, 22B, and 22C are out of axial alignment with one another). For some applications, each of circumferential frame segments 22A, 22B, and 22C of split-frame cardiac valve 20, 20F comprises two of tracks 36, which, when split-frame cardiac valve 20, 20F is in the radially expanded configuration, are disposed on opposite axially-oriented edges of the stent of the circumferential frame segment.

Reference is still made to FIGS. 8C-D. For some applications, at least two of sliders 38 (e.g., exactly two sliders (as shown) or exactly three sliders (configuration not shown)) couple each of pairs of tracks 36 together, such as described hereinabove with reference to FIGS. 1A-4B, mutatis mutandis.

Reference is still made to FIGS. 8C-D. For some applications, the two end circumferential frame segments 22A and 22C are identical to each other, and axially face in opposite directions, and the axially middle circumferential frame segment 22B is different from the two end circumferential frame segments 22A and 22C. For example, both tracks 36 of the axially middle circumferential frame segment 22B may have the same 24 length as each other, while each of the two end circumferential frame segments 22A and 22C has a longer track 36 and a shorter track 36, in order to enable the greater axial distance between these end segments when circumferential frame segments 22A, 22B, and 22C are out of axial alignment with one another, such as shown in FIGS. 8C-D.

Reference is made to FIGS. 6A, 6B, 7A-B, and 8A-B. For some applications, a number of circumferential frame segments 22 equals a number of tracks 36 of split-frame cardiac valve 20, 20C, 20D, 20E; in other words, split-frame cardiac valve 20, 20C, 20D, 20E comprises the same number of circumferential frame segments 22 as tracks 36. For example, the number may be two, such as shown in FIGS. 6A-B, or three, such as shown in FIGS. 7A-B and 8A-B.

Reference is made to FIGS. 6A and 7A-B. For some applications, each of circumferential frame segments 22 of split-frame cardiac valve 20, 20C, 20E c one of tracks 36, which, when split-frame cardiac valve 20, 20C, 20E is in the radially expanded configuration, is disposed on an axially-oriented edge 25 of stent 24 of the circumferential frame segment 22.

For some of these applications in which split-frame cardiac valve 20, 20C, 20E comprises sliders 38, each of sliders 38 is coupled to an axially-oriented edge 25 of the stent of one of the circumferential frame segments 22, opposite the axially-oriented edge 25 to which track 36 of the circumferential frame segment 22 is disposed, such as shown in FIGS. 6A and 7A-B. Each of sliders 38 is slidingly coupled to one of tracks 36 of one of the circumferential frame segments 22 to which slider 38 is not coupled.

Reference is made to FIG. 6B. For some applications, one of circumferential frame segments 22 of split-frame cardiac valve 20, 20D comprises two of tracks 36, which, when split-frame cardiac valve 20, 20D is in the radially expanded configuration, are disposed on opposite axially-oriented edges 25 of stent 24 of the circumferential frame segment 22.

For some of these applications in which split-frame cardiac valve 20, 20D comprises sliders 38, one of the circumferential frame segments 22 comprises two sliders 38, which, when split-frame cardiac valve 20, 20D is in the radially expanded configuration, are disposed on opposite axially-oriented edges 25 of stent 24 of the circumferential frame segment 22, such as shown in FIG. 6B. Each of sliders 38 is slidingly coupled to one of the tracks 36.

Reference is made to FIGS. 1A-8D. For some applications, some elements of split-frame cardiac valve 20 have one or more of the following dimensions:
each of tracks 36 has a length L1 of at least 1 cm, no more than 10 cm, and/or 1-10 cm (labeled in FIG. 1A), when circumferential frame segments 22 are axially aligned with one another so as to define tubular stent 32, tubular stent 32 has a segment-aligned length L2 of at least 1 cm, no more than 10 cm, and/or 1-10 cm, measured along central longitudinal axis 26 (labeled in FIG. 1B), when circumferential frame segments 22 are axially aligned with one another so as to define tubular stent 32, tubular stent 32 has a perimeter of at least 3 cm, no more than 20 cm, and/or 3-20 cm, measured around central longitudinal axis 26, for configurations in which tubular stent 32 is circularly cylindrical when circumferential frame segments 22 are axially aligned with one another so as to define tubular stent 32, tubular stent 32 has a diameter of at least 1 cm, no more than 7 cm, and/or 1-7 cm, the length L1 of each of tracks 36 is equal to at least 100%, no more than 150%, and/or 100%-150% cm of the segment-aligned length L2 of tubular stent 32, measured along central longitudinal axis 26 when circumferential frame segments 22 are axially aligned with one another so as to define tubular stent 32, when circumferential frame segments 22 are axially aligned with one another so as to define tubular stent 32, a ratio of (a) a perimeter of tubular stent 32, measured around central longitudinal axis 26, to (b) the segment-aligned length L2 of tubular stent 32, measured along central longitudinal axis 26, is at least 0.1, no more than 10, and/or 0.1-10, and/or for configurations in which tubular stent 32 is circularly cylindrical when circumferential frame segments 22 are axially aligned with one another so as to define tubular stent 32, a ratio of (a) a diameter of tubular stent 32 to (b) the segment-aligned length L2 of tubular stent 32, measured along central longitudinal axis 26, is at least 0.1, no more than 7, and/or 0.1-7.

Reference is still made to FIGS. 1A-8D. When circumferential frame segments 22 are axially aligned with one another so as to define tubular stent 32, tubular stent 32 has segment-aligned length L2, measured along central longitudinal axis 26, such as shown in FIGS. 1B, 2C, 4A-B, 5B-C, 7B, and 8B (labeled in FIG. 1B). When circumferential frame segments 22 are maximally out of axial alignment with one another, tubular stent 32 has a segment-non-aligned length L3, measured along central longitudinal axis 26, such as shown in FIGS. 1A, 2A, 3, 5A, 6A, 7A, and 8A (labeled in FIG. 1A).

For some applications, split-frame cardiac valve 20 is configured such that:
the segment-non-aligned length L3 equals at least the product of (a) the number of circumferential frame segments 22 and (b) the segment-aligned length L2, and/or
the segment-non-aligned length L3 equals at least 2 cm, no more than 20 cm, and/or 2-20 cm.

Reference is now made to FIG. 9, which is a schematic illustration of a cardiac valve system that comprises split-frame cardiac valve 20 and a delivery catheter 40, in accordance with an application of the present invention. Although FIG. 9 shows split-frame cardiac valve 20A by way of example, split-frame cardiac valves 20B, 20C, 20D, 20E, and 20F may also be deployed in this manner, mutatis mutandis, as may a split-frame cardiac valve 20 having more than three circumferential frame segments 22. For clarity of illustration, prosthetic leaflet(s) 30 are not shown.

Split-frame cardiac valve 20 is removably disposed in delivery catheter 40 in a radially compressed configuration such that the two or more circumferential frame segments 22A and 22B are at least partially non-axially-overlapping with one another. For some applications, split-frame cardiac valve 20 is removably disposed in delivery catheter 40 such that stents 24A and 24B are entirely non-axially-overlapping with one another.

This at least partially non-axially-overlapping disposition of split-frame cardiac valve 20 in delivery catheter 40 reduces the crossing profile of the valve compared to conventional prosthetic valves, thereby allowing the use of a lower-diameter delivery catheter. This disposition also reduces the stress on the two or more prosthetic leaflets when they are crimped within the delivery catheter.

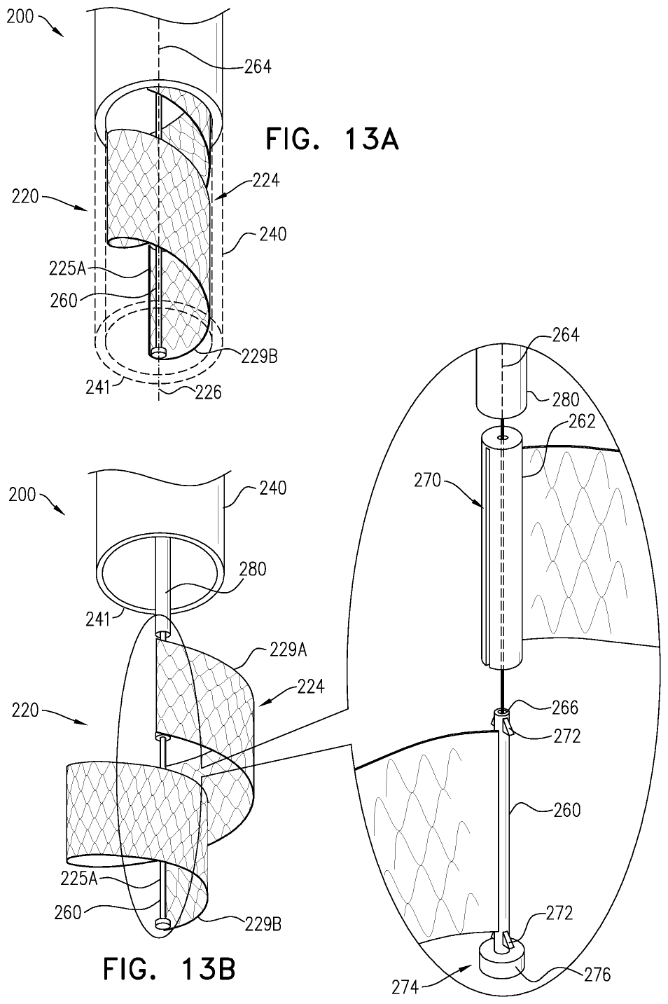

In some embodiments of the present invention, a method is provided that comprises advancing a delivery catheter toward a native cardiac valve annulus in a transcatheter procedure (e.g., a transapical or a transvascular procedure) while split-frame cardiac valve 20 is removably disposed in delivery catheter 40 such that the two or more circumferential frame segments 22 are at least partially non-axially-overlapping with one another. Stents 24 are anchored to the native cardiac valve annulus by sequentially deploying the two or more circumferential frame segments 22 from the delivery catheter, and axially aligning circumferential frame segments 22 with one another outside the delivery catheter, such that stents 24 collectively define tubular stent 32 that entirely surrounds central longitudinal axis 26, and the two or more prosthetic leaflets 30 are configured to assume open and closed states, and to coapt with one another when in the closed states. The implantation of split-frame cardiac valve 20 in the native cardiac valve annulus may be similar to that of split-frame cardiac valve 120 shown in FIG. 12C, mutatis mutandis.

For some applications, delivery catheter 40 is advanced toward the native cardiac valve annulus while split-frame cardiac valve 20 is removably disposed in the delivery catheter such that stents 24 are entirely non-axially-overlapping with one another.

As mentioned above, for some applications, each of circumferential frame segments 22A and 22B comprises two tracks 36 disposed on opposite axially-oriented edges 25 of the stent 24A and 24B of the circumferential frame segment 22A and 22B, and split-frame cardiac valve 20 comprises sliders 38, which slidingly couple pairs 42 of tracks 36 together. For some of these applications, after delivery from catheter 40, circumferential frame segments 22A and 22B are axially aligned with one another outside delivery catheter 40 by sliding pairs 42 of tracks 36 with respect to one another.

For some applications, the method further comprises, after circumferential frame segments 22A and 22B are axially aligned with one another, axially locking sliders 38 with respect to tracks 36 after axially aligning circumferential frame segments 22A and 22B with one another.

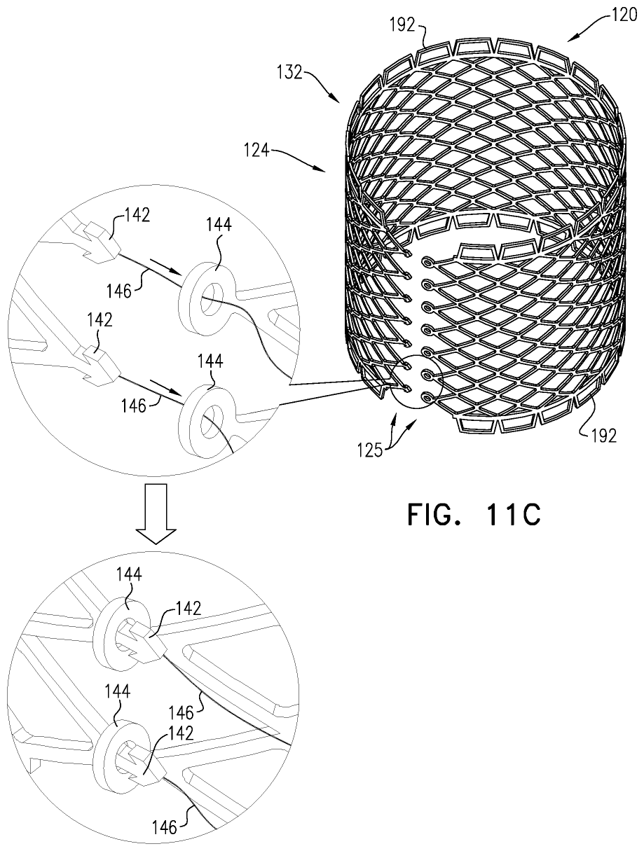

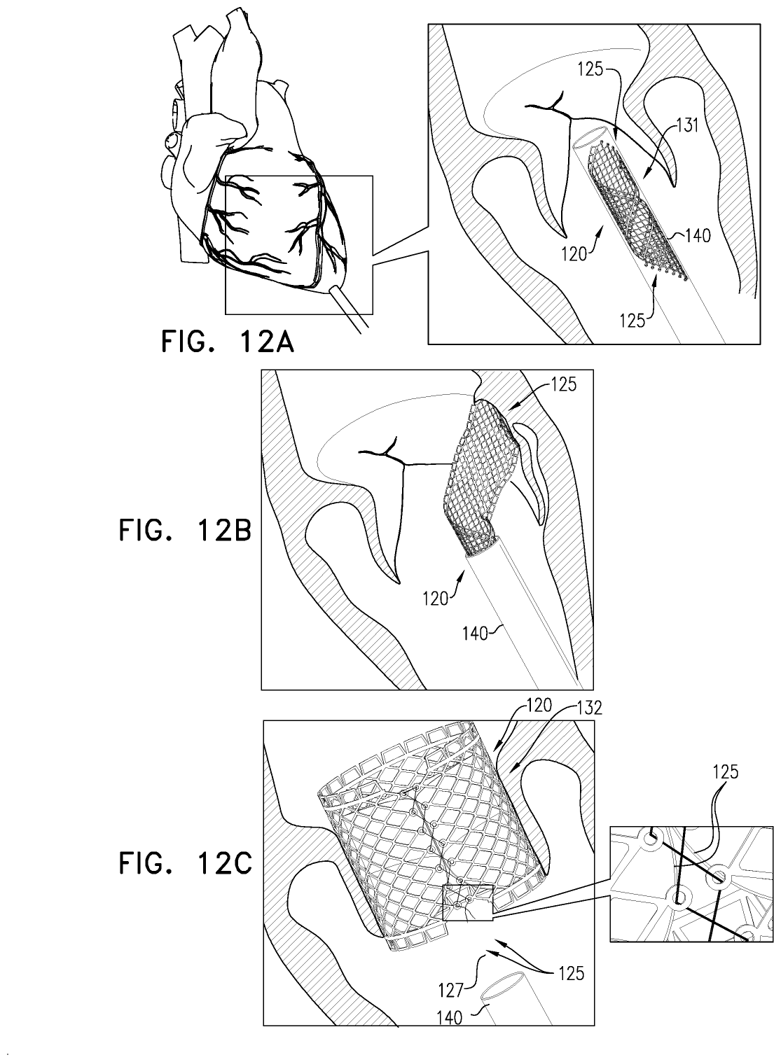

Reference is now made to FIGS. 10A-B, which are schematic illustrations of a split-frame cardiac valve 120, in accordance with an application of the present invention. FIGS. 10A-B (and FIGS. 11A-C and 12C, described hereinbelow) show split-frame cardiac valve 120 in a radially expanded configuration for anchoring to a native cardiac valve annulus.

Split-frame cardiac valve 120 comprises:
an axially-split tubular stent 124 having two free longitudinal edges 125 and two end edges 129; and two or more prosthetic leaflets 130, which are coupled to tubular stent 124.

When split-frame cardiac valve 120 is in the radially expanded configuration upon delivery from a delivery catheter 140, such as shown in FIGS. 10A-B, 11A-C, and 12C:
the two free longitudinal edges 125 are axially-oriented, i.e., extend axially along the stent parallel to a central longitudinal axis of tubular stent 124, and
the two end edges 129 are disposed at respective axial ends of tubular stent 124 and curve around the central longitudinal axis of tubular stent 124, i.e., are the edges of the bases of the stent.

Prosthetic leaflets 130 are shown in FIG. 10B, and, for clarity of illustration, are not shown in FIG. 10A.

Optionally, one or both ends of split-frame cardiac valve 120 (e.g., of axially-split tubular stent 124 thereof) are shaped so as to define anchors 192, which provide enhanced anchoring to the surrounding tissue, and may be optionally be configured to be tissue-penetrating (e.g., as barbs). Optionally, anchors 192 are oriented at least partially radially outward. Optionally, anchors 192 are coated by a material or covered by tissue to enhance endothelization.

Reference is additionally made to FIGS. 11A-C, which are schematic illustrations of split-frame cardiac valve 120 after the two free longitudinal axially-oriented edges 125 have been coupled together, in accordance with an application of the present invention. For example, the two free longitudinal axially-oriented edges 125 may be coupled together by being tied together using one or more sutures 127, such as shown in FIGS. 11A-B. Prosthetic leaflets 130 are shown in FIG. 11B, and, for clarity of illustration, are not shown in FIG. 11A or 11C.

Alternatively, for example, as shown in FIG. 11C, the two free longitudinal axially-oriented edges 125 may be coupled together by inserting a plurality of male anchors 142 (e.g., arrow-shaped or barbed), arranged on one of the longitudinal axially-oriented edges 125, into a corresponding plurality of female anchor receptacles 144, arranged on the other of the longitudinal axially-oriented edges 125. The male anchors become locked with respect to the female anchor receptacles upon insertion. For example, respective sutures 146 may be coupled to male anchors 142 passing through female anchor receptacles 144, in order to aid with insertion of male anchors 142 into female anchor receptacles 144.

Optionally, female anchor receptacles 144 are angled with respect to an external surface of axially-split tubular stent 124, e.g., with a 90-degree angle, to aid with insertion and coupling.

As shown in FIGS. 11A-C, split-frame cardiac valve 120 is configured such that when the two free longitudinal axially-oriented edges 125 are coupled together, axially-split tubular stent 124 assumes a tubular shape 132 in which the two or more prosthetic leaflets are configured to assume open and closed states, and to coapt with one another when in the closed states. For some applications, tubular shape 132 collectively defined by stents 24A and 24B is cylindrical, e.g., circularly cylindrical (such as shown in the figures) or non-circularly elliptically cylindrical (configuration not shown).

Reference is now made to FIGS. 12A-C, which are schematic illustrations of a method for deploying split-frame cardiac valve 120 in a native cardiac valve annulus 150, in accordance with an application of the present invention. For some applications, a cardiac valve system 110 is provided that comprises split-frame cardiac valve 120 and further comprises delivery catheter 140 in which split-frame cardiac valve 120 is removably disposed with the axially-split tubular stent 124 for transcatheter delivery, radially compressed in a three-dimensional spiral configuration 131, with one of the free longitudinal axially-oriented edges 125 disposed distal-most in delivery catheter 140 and the other of the free longitudinal axially-oriented edges 125 disposed proximal-most in delivery catheter 140, such as shown in FIG. 12A.

This three-dimensional spiral configuration 131 reduces the crossing profile of the valve compared to conventional prosthetic valves, thereby allowing the use of a lower-diameter delivery catheter. This configuration also reduces the stress on the two or more prosthetic leaflets when they are crimped within the delivery catheter.

The three-dimensional spiral configuration 131 may have a constant diameter, such as shown (in which case it may be considered a helical configuration), or may have a varying diameter (configuration not shown).

Reference is still made to FIGS. 12A-C. In some applications of the present invention, a method is provided that comprises advancing delivery catheter 140 toward native cardiac valve annulus 150 in a transcatheter procedure while split-frame cardiac valve 120 is removably disposed in delivery catheter 140 with the axially-split tubular stent 124 of split-frame cardiac valve 120 radially compressed in three-dimensional spiral configuration 131, with one of the two free longitudinal edges 125 of axially-split tubular stent 124 disposed distal-most in delivery catheter 140 and the other of the two free longitudinal edges 125 disposed proximal-most in delivery catheter 140, such as shown in FIG. 12A. The transcatheter procedure may be a transapical procedure (such as shown) or a transvascular procedure (configuration not shown).

Split-frame cardiac valve 120 is deployed from delivery catheter 140, as shown in FIGS. 12B-C. The two free longitudinal axially-oriented edges 125 are coupled together such that the axially-split tubular stent 124 assumes a tubular shape 132 in which the two or more prosthetic leaflets 130 are configured to assume open and closed states, and to coapt with one another when in the closed states. For some applications, the two free longitudinal axially-oriented edges 125 are coupled together by being tied together using two or more sutures 127, as shown in FIGS. 11A-B.

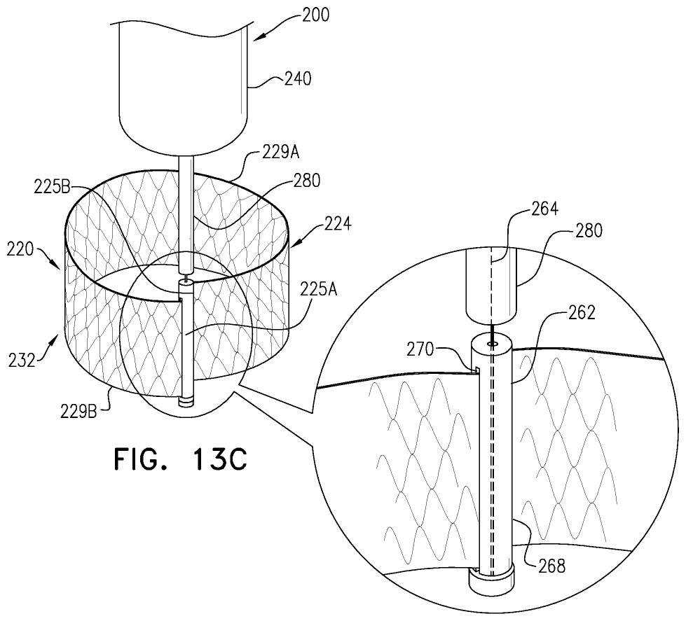

Reference is now made to FIGS. 13A-C, which are schematic illustrations of a cardiac valve system 200 and a method of using the system, in accordance with an application of the present invention. Cardiac valve system 200 comprises a split-frame cardiac valve 220 and a delivery catheter 240. Split-frame cardiac valve 220 may implement any of the features of split-frame cardiac valve 120, described hereinabove with reference to FIGS. 10A-12C, mutatis mutandis.

Split-frame cardiac valve 220 comprises:
an axially-split tubular stent 224 having first and second free longitudinal edges 225A and 225B and proximal and distal end edges 229A and 229B; and
two or more prosthetic leaflets, which are coupled to tubular stent 224; for clarity of illustration, the prosthetic leaflets are not shown in FIGS. 13A-C, but they may be generally similar to prosthetic leaflets 130 of split-frame cardiac valve 120 shown in FIGS. 10B and 11B, mutatis mutandis.

FIG. 13A shows split-frame cardiac valve 220 removably disposed in delivery catheter 240 with axially-split tubular stent 224 radially compressed in a rolled configuration defining more than one turn (typically at least 1.5 turns, e.g., 1.5-3 turns), in which first and second free longitudinal edges 225A and 225B are axially-oriented parallel to each other and to a central longitudinal axis 226 of delivery catheter 240, and first free longitudinal edge 225A is disposed more distally than second longitudinal edge 225B.

This rolled configuration reduces the crossing profile of the valve compared to conventional prosthetic valves, thereby allowing the use of a lower-diameter delivery catheter. This configuration also reduces the stress on the two or more prosthetic leaflets when they are crimped within the delivery catheter.

FIG. 13B shows split-frame cardiac valve 220 after deployment thereof from a distal end 241 of delivery catheter 240, in a partially radially expanded configuration before the completion of a transition to a radially expanded configuration.

FIG. 13C shows split-frame cardiac valve 220 in the radially expanded configuration upon deployment from distal end 241 of delivery catheter 240. Split-frame cardiac valve 220 is configured such that when split-frame cardiac valve 220 is in the radially expanded configuration and first and second free longitudinal edges 225A and 225B are coupled together, axially-split tubular stent 224 assumes tubular shape 232 in which:
first and second free longitudinal edges 225A and 225B are axially-oriented and proximal and distal end edges 229A and 229B are disposed at respective axial ends of tubular stent 224, and
the prosthetic leaflets are configured to assume open and closed states, and to coapt with one another when in the closed states.

For some applications, such as shown in FIG. 13A, split-frame cardiac valve 220 is removably disposed within delivery catheter 240 with axially-split tubular stent 224 radially compressed in the rolled configuration in which a proximal end (the upper end in the figure) of first free longitudinal edge 225A is disposed more distally than a distal end (the lower end in the figure) of second longitudinal edge 225B. For example, a distance between the proximal end of first free longitudinal edge 225A and the distal end of second longitudinal edge 225B may equals at least 25% of a length of first free longitudinal edge 225A, such as at least 50%, e.g., at least 75%, such as at least 100% of the length of first free longitudinal edge 225A.

For some applications, as shown in FIGS. 13A-C, split-frame cardiac valve 220 further comprises a shaft 260, which is coupled to first free longitudinal edge 225A; and a shaft receptacle 262, which is coupled to second longitudinal edge 225B. Shaft receptacle 262 is configured to slidingly accept shaft 260, such that upon insertion of shaft 260 into shaft receptacle 262 after deployment of split-frame cardiac valve 220 from distal end 241 of delivery catheter 240, such as shown in FIG. 13B, first and second free longitudinal edges 225A and 225B are coupled together by shaft 260 and shaft receptacle 262, such as shown in FIG. 13C. For example, a length of shaft 260 may be 25%-125% of the length of first free longitudinal edge 225A, and/or a length of shaft receptacle 262 may be no more than 125% of the length of first free longitudinal edge 225B.

For some of these applications, cardiac valve system 200 further comprises a deployment wire 264, which is removably coupled to a proximal end 266 of shaft 260 (labeled in FIG. 13B) and passes through shaft receptacle 262 and delivery catheter 240 when split-frame cardiac valve 220 is removably disposed within delivery catheter 240, such as shown in FIG. 13A. Deployment wire 264 may be used as described hereinbelow.

For some applications, such as shown in FIGS. 13A-C, shaft receptacle 262 is at least partially cylindrical. For some of these applications, shaft receptacle 262 is shaped as a partial cylinder 268 that defines a longitudinal slot 270 therealong. Alternatively, for some of these applications, shaft receptacle 262 is entirely cylindrical (configuration not shown).

For some applications, shaft 260 is shaped so as to define one or more couplers 272 (e.g., protrusions, such as barbs or arrows) that are configured to engage shaft receptacle 262 to prevent decoupling of shaft 260 from shaft receptacle 262 after insertion therein.

For some applications, a distal end 274 of shaft 260 comprises a stopper 276 (labeled in FIG. 13B), which limits advancement of shaft 260 into shaft receptacle 262.

As shown in FIGS. 11A-C, split-frame cardiac valve 120 is configured such that when the two free axially-oriented edges 125 are coupled together, axially-split tubular stent 124 assumes a tubular shape 132 in which the two or more prosthetic leaflets are configured to assume open and closed states, and to coapt with one another when in the closed states. For some applications, tubular shape 132 collectively defined by stents 24A and 24B is cylindrical, e.g., circularly cylindrical (such as shown in the figures) or non-circularly elliptically cylindrical (configuration not shown).

Reference is still made to FIGS. 13A-C. In some applications, a method is provided that comprises advancing delivery catheter 240 toward a native cardiac valve annulus in a transcatheter procedure while split-frame cardiac valve 220 is removably disposed in delivery catheter 240 for transcatheter delivery, with axially-split tubular stent 224 radially compressed in the rolled configuration defining more than one turn, in which first and second free longitudinal edges of tubular stent 224 axially-oriented parallel to each other and central longitudinal axis 226 of delivery catheter 240, and first free longitudinal edge 225A of tubular stent 224 is disposed more distally than second free longitudinal edge 225B of tubular stent 224. A portion of this method is shown in FIG. 13A, while the advancing may be similar to the method described hereinabove with reference to FIG. 12A. The transcatheter procedure may be a transapical procedure (such as shown in FIG. 12A) or a transvascular procedure (configuration not shown).

As shown in FIGS. 13B-C, split-frame cardiac valve 220 is deployed from distal end 241 of delivery catheter 240, such that split-frame cardiac valve 220 assumes a radially expanded configuration, and first and second free longitudinal edges 225A and 225B are coupled together such that axially-split tubular stent 224 assumes tubular shape 232 in which first and second free longitudinal edges 225A and 225B are axially-oriented and proximal and distal end edges 229A and 229B of tubular stent 224 are disposed at respective axial ends of tubular stent 224, and the prosthetic leaflets are configured to assume open and closed states, and to coapt with one another when in the closed states (as mentioned above, the prosthetic leaflets are not shown in FIGS. 13A-C, but they may be generally similar to prosthetic leaflets 130 of split-frame cardiac valve 120 shown in FIGS. 10B and 11B, mutatis mutandis).

For some applications in which split-frame cardiac valve 220 further comprises shaft 260 and shaft receptacle 262, as described above, first and second free longitudinal edges 225A and 225B are coupled together by sliding shaft 260 into shaft receptacle 262 such that first and second free longitudinal edges 225A and 225B are coupled together by shaft 260 and shaft receptacle 262. For example, the sliding may be performed by moving a delivery tube 280 of cardiac valve system 200 and deployment wire 264 with respect to each other, e.g., by distally pushing delivery tube 280 and/or proximally pulling deployment wire 264. Typically, deployment wire 264 is decoupled after shaft 260 has been coupled to receptacle 262, such as by unscrewing the deployment wire or using a quick release mechanism.

For some applications, coupling together first and second free longitudinal edges 225A and 225B further comprises, after sliding shaft 260 into shaft receptacle 262, further coupling together first and second free longitudinal edges 225A and 225B using one or more coupling elements selected from the group consisting of: one or more sutures and one or more anchors, for example using the techniques described hereinabove with reference to FIGS. 11A-B or FIG. 11C, mutatis mutandis.

Alternatively, for some applications in which sliding shaft 260 and shaft receptacle 262 are not provided, first and second free longitudinal edges 225A and 225B are coupled together using one or more coupling elements selected from the group consisting of: one or more sutures and one or more anchors, for example using the techniques described hereinabove with reference to FIGS. 11A-B or FIG. 11C, mutatis mutandis.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

What is claimed is:

1.-48. (canceled)

48. Apparatus comprising
A cardiac valve system comprising:
a split-frame cardiac valve, which comprises:
an axially-split tubular stent having two free longitudinal edges and two end edges; and
two or more prosthetic leaflets, which are coupled to the tubular stent; and
a delivery catheter in which the split-frame cardiac valve is removably disposed with the axially-split tubular stent radially compressed in a three-dimensional spiral configuration, with one of the free longitudinal edges disposed distal-most in the delivery catheter and the other of the free longitudinal edges disposed proximal-most in the delivery catheter,
wherein the split-frame cardiac valve is configured such that when the split-frame cardiac valve is in a radially expanded configuration upon delivery from the delivery catheter and the two free longitudinal edges are coupled together, the axially-split tubular stent assumes a tubular shape in which:
the two free longitudinal edges are axially-oriented and the two end edges are disposed at respective axial ends of the tubular stent, and the prosthetic leaflets are configured to assume open and closed states, and to coapt with one another when in the closed states.

49. A method comprising:
advancing a delivery catheter toward a native cardiac valve annulus in a transcatheter procedure while a split-frame cardiac valve is removably disposed in the delivery catheter with an axially-split tubular stent of the split-frame cardiac valve radially compressed in a three-dimensional spiral configuration, with one of two free longitudinal edges of the axially-split tubular stent disposed distal-most in the delivery catheter and the other of the two free longitudinal edges disposed proximal-most in the delivery catheter, wherein the split-frame cardiac valve further comprises two or more prosthetic leaflets, which are coupled to the tubular stent;
deploying the split-frame cardiac valve from the delivery catheter, such that the split-frame cardiac valve assumes a radially expanded configuration; and
coupling together the two free axially-oriented edges such that the axially-split tubular stent assumes a tubular shape in which the two free longitudinal edges are axially-oriented and two end edges of the axially-split tubular stent are disposed at respective axial ends of the tubular stent, and the prosthetic leaflets are configured to assume open and closed states, and to coapt with one another when in the closed states.

50. Apparatus comprising
A cardiac valve system comprising:
a split-frame cardiac valve, which comprises:
an axially-split tubular stent having first and second free longitudinal edges and proximal and distal end edges; and
two or more prosthetic leaflets, which are coupled to the tubular stent; and
a delivery catheter in which the split-frame cardiac valve is removably disposed with the axially-split tubular stent radially compressed in a rolled configuration defining more than one turn, in which the first and the second free longitudinal edges are axially-oriented parallel to each other and to a central longitudinal axis of the delivery catheter, and the first free longitudinal edge is disposed more distally than the second free longitudinal edge, wherein the split-frame cardiac valve is configured such that when the split-frame cardiac valve is in a radially expanded configuration upon deployment from a distal end of the delivery catheter and the first and the second free longitudinal edges are coupled together, the axially-split tubular stent assumes a tubular shape in which:
the first and the second free longitudinal edges are axially-oriented and the proximal and the distal end edges are disposed at respective axial ends of the tubular stent, and the prosthetic leaflets are configured to assume open and closed states, and to coapt with one another when in the closed states.

51. The apparatus according to claim 50, wherein the split-frame cardiac valve is removably disposed within the delivery catheter with the axially-split tubular stent radially compressed in the rolled configuration in which a proximal end of the first free longitudinal edge is disposed more distally than a distal end of the second free longitudinal edge.

52. The apparatus according to claim 51, wherein the split-frame cardiac valve is removably disposed within the delivery catheter with the axially-split tubular stent radially compressed in the rolled configuration in which a distance between the proximal end of the first free longitudinal edge and the distal end of the second free longitudinal edge equals at least 25% of a length of the first free longitudinal edge.

53. The apparatus according to claim 51, wherein the split-frame cardiac valve further comprises:
a shaft, which is coupled to the first free longitudinal edge; and
a shaft receptacle, which is coupled to the second free longitudinal edge, and is configured to slidingly accept the shaft, such that upon insertion of the shaft into the shaft receptacle after deployment of the split-frame cardiac valve from the distal end of the delivery catheter, the first and the second free longitudinal edges are coupled together by the shaft and the shaft receptacle.

54. The apparatus according to claim 53, further comprising a deployment wire, which is removably coupled to a proximal end of the shaft and passes through the shaft receptacle and the delivery catheter when the split-frame cardiac valve is removably disposed within the delivery catheter.

55. The apparatus according to claim 53, wherein the shaft receptacle is at least partially cylindrical.

56. The apparatus according to claim 53, wherein the shaft receptacle is shaped as a partial cylinder that defines a longitudinal slot therealong.

57. The apparatus according to claim 53, wherein the shaft is shaped so as to define one or more couplers that are configured to engage the shaft receptacle to prevent decoupling of the shaft from the shaft receptacle after insertion therein.

58. The apparatus according to claim 53, wherein a distal end of the shaft comprises a stopper, which limits advancement of the shaft into the shaft receptacle.

59. A method comprising:
advancing a delivery catheter toward a native cardiac valve annulus in a transcatheter procedure while a split-frame cardiac valve is removably disposed in the delivery catheter with an axially-split tubular stent of the split-frame cardiac valve radially compressed in a rolled configuration defining more than one turn, in which first and second free longitudinal edges of the tubular stent axially-oriented parallel to each other and to a central longitudinal axis of the delivery catheter, and a first free longitudinal edge of the tubular stent is disposed more distally than a second free longitudinal edge of the tubular stent, wherein the split-frame cardiac valve further comprises two or more prosthetic leaflets, which are coupled to the tubular stent;
deploying the split-frame cardiac valve from a distal end of the delivery catheter, such that the split-frame cardiac valve assumes a radially expanded configuration; and
coupling together the first and the second free longitudinal edges such that the axially-split tubular stent assumes a tubular shape in which the first and the second free longitudinal edges are axially-oriented and proximal and distal end edges of the tubular stent are disposed at respective axial ends of the tubular stent, and the prosthetic leaflets are configured to assume open and closed states, and to coapt with one another when in the closed states.

60. The method according to claim 59, wherein advancing the delivery catheter comprises advancing the delivery catheter while the split-frame cardiac valve is removably disposed within the delivery catheter with the axially-split tubular stent radially compressed in the rolled configuration in which a proximal end of the first free longitudinal edge is disposed more distally than a distal end of the second free longitudinal edge.

61. The method according to claim 60, wherein advancing the delivery catheter comprises advancing the delivery catheter while the split-frame cardiac valve is removably disposed within the delivery catheter with the axially-split tubular stent radially compressed in the rolled configuration in which a distance between the proximal end of the first free longitudinal edge and the distal end of the second free longitudinal edge equals at least 25% of a length of the first free longitudinal edge.

62. The method according to claim 60, wherein the split-frame cardiac valve further comprises (a) a shaft, which is coupled to the first free longitudinal edge, and (b) a shaft receptacle, which is coupled to the second free longitudinal edge, and wherein coupling together the first and the second free longitudinal edges comprises sliding the shaft into the shaft receptacle such that the first and the second free longitudinal edges are coupled together by the shaft and the shaft receptacle.

63. The method according to claim 62, wherein coupling together the first and the second free longitudinal edges further comprises, after sliding the shaft into the shaft receptacle, further coupling together the first and the second free longitudinal edges using one or more coupling elements selected from the group consisting of: one or more sutures and one or more anchors.

64. The method according to claim 62, wherein sliding the shaft into the shaft receptacle comprises pulling, in a proximal direction, a deployment wire that is removably coupled to a proximal end of the shaft and passes through the shaft receptacle and the delivery catheter when the split-frame cardiac valve is removably disposed within the delivery catheter.

65. The method according to claim 62, wherein the shaft receptacle is at least partially cylindrical.

66. The method according to claim 62, wherein the shaft receptacle is shaped as a partial cylinder that defines a longitudinal slot therealong.

67. The method according to claim 62, wherein the shaft is shaped so as to define one or more couplers that are configured to engage the shaft receptacle to prevent decoupling of the shaft from the shaft receptacle after insertion therein.

68. The method according to claim 62, wherein a distal end of the shaft comprises a stopper, which limits advancement of the shaft into the shaft receptacle.

69. The method according to claim 59, wherein coupling together the first and the second free longitudinal edges comprises coupling together the first and the second free longitudinal edges using one or more coupling elements selected from the group consisting of: one or more sutures and one or more anchors.
